SEQUENCE LISTING (162 sequences)

SEQ ID NO: 1 - protein, 7 aa, Unknown
  Description of Unknown Removable protecting group sequence
MENLYFQ

SEQ ID NO: 2 - protein, 6 aa, Unknown
  Description of Unknown TEV protease cleavage site sequence
ENLYFQ

SEQ ID NO: 3 - protein, 6 aa, Artificial Sequence
  Description of Artificial Sequence Synthetic peptide
LFVQEL

SEQ ID NO: 4 - DNA, 990 bp, Artificial Sequence
  Description of Artificial Sequence Synthetic polynucleotide
atgcagatttttgtgaagactttaacaggtaagacgattaccctggaggtggagtcctcggacaccatcgataatgtaaaatcaaaaatccaagataaggaaggaatccctccagaccagcaacgtctgattttcgcaggtaaacaactggaggatggtcgcacgctttcggactacaacatccagaaagaatctacccttcatttggttctgcgtctgcgtggaggatagttgtttgtgcaggagcttgcatccaagggcgaggagctctttactggcgtagtaccaattctcgtagagctcgatggcgatgtaaatggccataagttttccgtacgcggcgagggcgagggcgatgcaactaacggcaagctcactctcaagtttatttgtactactggcaagctcccagtaccatggccaactctcgtaactactctgacctatggcgtacaatgtttttcccgctatccagatcacatgaagcaacatgatttttttaagtccgcaatgccagagggctatgtacaagagcgcactattagctttaaggatgatggcacctataagactcgcgcagaggtaaagtttgagggcgatactctcgtaaatcgcattgagctcaagggcattgattttaaggaggatggcaatattctcggccataagctggagtataatttcaattcccataatgtatacattaccgcagataagcaaaagaatggcattaaggcgaattttaagattcgccataatgtggaggatggctccgtacaactcgcagatcattatcaacaaaatactccaattggcgatggcccagtactcctcccagataatcattatctctccactcaatccgtgctctccaaagatccaaatgagaagcgcgatcacatggtactcctggagtttgtaactgcagcaggcattactcatggcatggatgagctctataagctcgagcaccaccaccaccaccactaa

SEQ ID NO: 5 - DNA, 312 bp, Artificial Sequence
  Description of Artificial Sequence Synthetic polynucleotide
atgtctgatagtcctgttgacttaaaacccaagcctaaagtcaagcccaaattagaacgcccaaaactttacaaagtcatgttattgaatgatgattatacaccacgcgaatttgtgacggtagtccttaaagcggtgtttcgtatgtcagaggacactggtcgccgtgtaatgatgacagcacatcgttttggttcggcggtggtggtcgtttgtgaacgtgacattgcagagacgaaagccaaggaggcgaccgacttggggaaggaagcaggttttcctttgatgttcacgactgagcccgaggagtaa

SEQ ID NO: 6 - DNA, 300 bp, Artificial Sequence
  Description of Artificial Sequence Synthetic polynucleotide
gttatgcactacgatggtgttgacgtttacgttggtggtatggaacagcgtaaaatccacatgctggcgcgtgaactgctgccgaaaaaagttgtttgcatccacaacccggttctgaccggtctggacggtgaaggtaaaatgtcttcttctaaaggtaacttcatcgcggttgacgactctccggaagaaatccgtgcgaaaatcaaaaaagcgtactgcccggcgggtgttgttgaaggtaacccgatcatggaaatcgcgaaatacttcctggaatacccgctgaccatcaaaggt

SEQ ID NO: 7 - DNA, 2151 bp, Artificial Sequence
  Description of Artificial Sequence Synthetic polynucleotide
atggggagtgggtctttcattgctgggcttgtcaacgatggtaatacgtgttttatgaactcggttcttcagtcccttgctagtagccgtgaacttatggagtttttggataataatgtaatccgtacatatgaagaaattgaacagaacgagcacaatgaggaaggtaatggccaagagagcgcacaagatgaggcaactcacaaaaaaaacactcgcaagggaggtaaggtctatgggaagcataaaaagaaattaaaccgcaaatcttctagcaaggaagacgaagaaaagtcgcaagaaccagacattacgttttcggtggcgttgcgtgatctgctgagcgcattaaatgctaagtattatcgcgacaaaccctactttaagactaactctttattaaaagcgatgagcaagtccccgcgcaaaaatatcttgcttgggtacgatcaagaagacgctcaggaattttttcaaaacattcttgcggagttagaatctaatgtcaagtcgttaaacacagaaaagcttgatactacaccggtagccaagtccgaacttccagacgatgctctggttggccaattaaaccttggtgaggtaggcaccgtgtacattcccacagaacaaattgaccccaattcgattttacatgacaaatcgattcaaaactttaccccctttaaactgatgaccccgttggatgggatcacggctgagcgcatcggctgcctgcaatgcggagagaacgggggaattcgctacagtgttttcagcggattaagtttgaacctgccgaatgaaaatattggaagcactcttaaactgtcccagttactgtccgattggtcgaaacccgagattatcgagggtgttgaatgcaaccgttgcgctttaacagctgcgcactcacacttgtttggccaattaaaggagtttgagaagaaacctgaaggctcgattcccgaaaaacttattaatgccgtaaaggaccgcgtgcaccagatcgaagaggtcttggcaaagccggttatcgacgatgaagattataaaaaattgcatactgcgaatatggtccgcaagtgttcaaaaagtaaacaaattcttatctctcgtccaccacctttgttgtctattcatatcaaccgctctgttttcgacccgcgcacctacatgattcgcaagaacaactccaaggttttgttcaagtcacgcttgaacctggcaccctggtgctgtgatatcaacgaaatcaatcttgacgcacgccttccgatgtcgaagaaggaaaaagcagctcaacaagattcttctgaagacgagaacattggcggagagtactatactaaattgcatgaacgttttgagcaggagtttgaagattctgaagaagagaaggaatacgatgatgcagagggtaattatgcatcgcattataaccataccaaggacatctccaactacgatccattgaatggagaagtcgacggtgtgacttccgatgatgaggatgaatacattgaagagacagacgcgttggggaataccatcaaaaaacgtattattgaacactccgacgtggagaacgaaaacgtgaaggataatgaagaacttcaggagatcgataacgttagcttggatgagccaaaaattaatgtcgaggaccagcttgaaacgagttctgatgaggaagacgttattcctgctccacccatcaactacgctcgcagctttagtacggtcccagcgacccctttaacttactctttgcgcagcgtcatcgtgcactatgggactcacaactacggacattatattgcatttcgcaagtatcgtggatgttggtggcgcatctccgatgagacggtctatgtggtagatgaggccgaagtactgtcaacaccgggggtatttatgcttttctacgagtatgatttcgacgaggagaccggaaaaatgaaagacgacttagaagctatccagagcaataatgaggaagatgacgagaaagaacaggaacagaagggtgtccaggagccaaaagaatcccaggagcaaggcgaaggcgaagaacaagaagaagggcaagagcaaatgaaatttgagcgtacggaggatcatcgcgacatttcagggaaggatgtgaattaa

SEQ ID NO: 8 - DNA, 23 bp, Artificial Sequence
  Description of Artificial Sequence Synthetic oligonucleotide
ccattattatcatgacattaacc

SEQ ID NO: 9 - DNA, 20 bp, Artificial Sequence
  Description of Artificial Sequence Synthetic oligonucleotide
ggatttgtcctactcaggag

SEQ ID NO: 10 - DNA, 18 bp, Artificial Sequence
  Description of Artificial Sequence Synthetic oligonucleotide
ctttttatcgcaactctc

SEQ ID NO: 11 - DNA, 41 bp, Artificial Sequence
  Description of Artificial Sequence Synthetic oligonucleotide
ttaaagaggagaaattaactatgcagatttttgtgaagact

SEQ ID NO: 12 - DNA, 40 bp, Artificial Sequence
  Description of Artificial Sequence Synthetic oligonucleotide
agctcctcgcccttggatgcaagctcctgcacaaacaagt

SEQ ID NO: 13 - DNA, 40 bp, Artificial Sequence
  Description of Artificial Sequence Synthetic oligonucleotide
cagggaaggatgtgaattaataagtcgaccatcatcatca

SEQ ID NO: 14 - DNA, 42 bp, Artificial Sequence
  Description of Artificial Sequence Synthetic oligonucleotide
atgaaagacccactccccatagatctaattcctcctgttagc

SEQ ID NO: 15 - DNA, 40 bp, Artificial Sequence
  Description of Artificial Sequence Synthetic oligonucleotide
taacaggaggaattagatctatggggagtgggtctttcat

SEQ ID NO: 16 - DNA, 40 bp, Artificial Sequence
  Description of Artificial Sequence Synthetic oligonucleotide
tcaagcgtgacttgaacaaaaccttggagttgttcttgcg

SEQ ID NO: 17 - DNA, 40 bp, Artificial Sequence
  Description of Artificial Sequence Synthetic oligonucleotide
cgcaagaacaactccaaggttttgttcaagtcacgcttga

SEQ ID NO: 18 - DNA, 41 bp, Artificial Sequence
  Description of Artificial Sequence Synthetic oligonucleotide
tgatgatgatggtcgacttattaattcacatccttccctga

SEQ ID NO: 19 - DNA, 40 bp, Artificial Sequence
  Description of Artificial Sequence Synthetic oligonucleotide
tgcgtctgcgtggaggatagttgtttgtgcaggagcttgc

SEQ ID NO: 20 - DNA, 37 bp, Artificial Sequence
  Description of Artificial Sequence Synthetic oligonucleotide
aagctcctgcacaaacaactatcctccacgcagacgc

SEQ ID NO: 21 - DNA, 20 bp, Artificial Sequence
  Description of Artificial Sequence Synthetic oligonucleotide
gcttgggtacgatcaagaag

SEQ ID NO: 22 - DNA, 21 bp, Artificial Sequence
  Description of Artificial Sequence Synthetic oligonucleotide
ccttggtatggttataatgcg

SEQ ID NO: 23 - DNA, 40 bp, Artificial Sequence
  Description of Artificial Sequence Synthetic oligonucleotide
cagggaaggatgtgaattaaaagcttgatgggggatccca

SEQ ID NO: 24 - DNA, 45 bp, Artificial Sequence
  Description of Artificial Sequence Synthetic oligonucleotide
atgaaagacccactccccatggtacctttctcctctttaatgaat

SEQ ID NO: 25 - DNA, 40 bp, Artificial Sequence
  Description of Artificial Sequence Synthetic oligonucleotide
ttaaagaggagaaaggtaccatggggagtgggtctttcat

SEQ ID NO: 26 - DNA, 41 bp, Artificial Sequence
  Description of Artificial Sequence Synthetic oligonucleotide
tgggatcccccatcaagcttttaattcacatccttccctga

SEQ ID NO: 27 - DNA, 45 bp, Artificial Sequence
  Description of Artificial Sequence Synthetic oligonucleotide
taaagaggagaaaggtaccatgcagatttttgtgaagactttaac

SEQ ID NO: 28 - DNA, 40 bp, Artificial Sequence
  Description of Artificial Sequence Synthetic oligonucleotide
tgggatcccccatcaagcttttagtggtggtggtggtggt

SEQ ID NO: 29 - DNA, 40 bp, Artificial Sequence
  Description of Artificial Sequence Synthetic oligonucleotide
accaccaccaccaccactaaaagcttgatgggggatccca

SEQ ID NO: 30 - DNA, 45 bp, Artificial Sequence
  Description of Artificial Sequence Synthetic oligonucleotide
gtcttcacaaaaatctgcatggtacctttctcctctttaatgaat

SEQ ID NO: 31 - DNA, 74 bp, Artificial Sequence
  Description of Artificial Sequence Synthetic oligonucleotide
ttacgggctaattacaggcagaaatgcgtgatgtgtgccacacttgttgatccctatcagtgatagagattgac

SEQ ID NO: 32 - DNA, 74 bp, Artificial Sequence
  Description of Artificial Sequence Synthetic oligonucleotide
ccagcgggctaactttcctcgccggaagagtggttaacaaaatagtaacgtcaccgacaaacaacagataaaac

SEQ ID NO: 33 - DNA, 21 bp, Artificial Sequence
  Description of Artificial Sequence Synthetic oligonucleotide
ccaaagtgagttgagtataac

SEQ ID NO: 34 - DNA, 20 bp, Artificial Sequence
  Description of Artificial Sequence Synthetic oligonucleotide
tttctccttattatcaatgc

SEQ ID NO: 35 - DNA, 90 bp, Artificial Sequence
  Description of Artificial Sequence Synthetic oligonucleotide
gccgcagcaagccaaagtgagttgagtataacgcaaatttgctactggtccgatgggtgcaatggtctgaattacgggctaattacaggc

SEQ ID NO: 36 - DNA, 88 bp, Artificial Sequence
  Description of Artificial Sequence Synthetic oligonucleotide
aacgcaatcgcaaccgctaaaccactggccatgtgcacgagtttcattcatttctccttattatcaatgcaccagcgggctaactttc

SEQ ID NO: 37 - DNA, 90 bp, Artificial Sequence
  Description of Artificial Sequence Synthetic oligonucleotide
taaagagctcctcgcccttggatgcaagctcctgcacaaacaacgatcctccacgcagacgcagaaccaaatgaagggtagattctttct

SEQ ID NO: 38 - DNA, 17 bp, Artificial Sequence
  Description of Artificial Sequence Synthetic oligonucleotide
cgtctgcgtggaggatc

SEQ ID NO: 39 - DNA, 17 bp, Artificial Sequence
  Description of Artificial Sequence Synthetic oligonucleotide
cgtctgcgtggaggata

SEQ ID NO: 40 - DNA, 40 bp, Artificial Sequence
  Description of Artificial Sequence Synthetic oligonucleotide
ttctgacccatcgtaattaaaagcttgatgggggatccca

SEQ ID NO: 41 - DNA, 44 bp, Artificial Sequence
  Description of Artificial Sequence Synthetic oligonucleotide
tggtatatctccttttattattaattcacatccttccctgaaat

SEQ ID NO: 42 - DNA, 45 bp, Artificial Sequence
  Description of Artificial Sequence Synthetic oligonucleotide
gtgaattaataataaaaggagatataccatgtcatacagcggcga

SEQ ID NO: 43 - DNA, 43 bp, Artificial Sequence
  Description of Artificial Sequence Synthetic oligonucleotide
tgggatcccccatcaagcttttaattacgatgggtcagaatcg

SEQ ID NO: 44 - DNA, 41 bp, Artificial Sequence
  Description of Artificial Sequence Synthetic oligonucleotide
ctgccaacttactgatttagtgtatgatggtgtttttgagg

SEQ ID NO: 45 - DNA, 41 bp, Artificial Sequence
  Description of Artificial Sequence Synthetic oligonucleotide
gccgcttagttagccgtgcaaacttatatcgtatggggctg

SEQ ID NO: 46 - DNA, 40 bp, Artificial Sequence
  Description of Artificial Sequence Synthetic oligonucleotide
agccccatacgatataagtttgcacggctaactaagcggc

SEQ ID NO: 47 - DNA, 45 bp, Artificial Sequence
  Description of Artificial Sequence Synthetic oligonucleotide
ctcaaaaacaccatcatacactaaatcagtaagttggcagcatca

SEQ ID NO: 48 - DNA, 40 bp, Artificial Sequence
  Description of Artificial Sequence Synthetic oligonucleotide
tgtgtacgctagaaaaagcctaaaagcttgatgggggatc

SEQ ID NO: 49 - DNA, 45 bp, Artificial Sequence
  Description of Artificial Sequence Synthetic oligonucleotide
gttcgttttacccatggtatatctccttttattattaattcacat

SEQ ID NO: 50 - DNA, 40 bp, Artificial Sequence
  Description of Artificial Sequence Synthetic oligonucleotide
ataataaaaggagatataccatgggtaaaacgaacgactg

SEQ ID NO: 51 - DNA, 40 bp, Artificial Sequence
  Description of Artificial Sequence Synthetic oligonucleotide
gatcccccatcaagcttttaggctttttctagcgtacaca

SEQ ID NO: 52 - DNA, 50 bp, Artificial Sequence
  Description of Artificial Sequence Synthetic oligonucleotide
tactgtttctccatacccgtttttttgggctaacaggaggaattagatct

SEQ ID NO: 53 - DNA, 23 bp, Artificial Sequence
  Description of Artificial Sequence Synthetic oligonucleotide
agatctaattcctcctgttagcc

SEQ ID NO: 54 - DNA, 90 bp, Artificial Sequence
  Description of Artificial Sequence Synthetic oligonucleotide
accccatgagtgcaatagaaaatttcgacgcccatacgcccatgatgcagcagtgatagtcgctgaaagcccagcatcccgagatcctgc

SEQ ID NO: 55 - DNA, 90 bp, Artificial Sequence
  Description of Artificial Sequence Synthetic oligonucleotide
accccatgagtgcaatagaaaatttcgacgcccatacgcccatgatgcagcagtatctcaggctgaaagcccagcatcccgagatcctgc

SEQ ID NO: 56 - DNA, 22 bp, Artificial Sequence
  Description of Artificial Sequence Synthetic oligonucleotide
ccatgatgcagcagtatctcag

SEQ ID NO: 57 - DNA, 22 bp, Artificial Sequence
  Description of Artificial Sequence Synthetic oligonucleotide
ccatgatgcagcagtgatagtc

SEQ ID NO: 58 - DNA, 20 bp, Artificial Sequence
  Description of Artificial Sequence Synthetic oligonucleotide
aggttgtcctgacgctcctg

SEQ ID NO: 59 - DNA, 28 bp, Artificial Sequence
  Description of Artificial Sequence Synthetic oligonucleotide
gtataatttcaattcccataatgtatag

SEQ ID NO: 60 - DNA, 28 bp, Artificial Sequence
  Description of Artificial Sequence Synthetic oligonucleotide
gtataatttcaattcccataatgtatac

SEQ ID NO: 61 - DNA, 21 bp, Artificial Sequence
  Description of Artificial Sequence Synthetic oligonucleotide
ctcgagcttatagagctcatc

SEQ ID NO: 62 - DNA, 90 bp, Artificial Sequence
  Description of Artificial Sequence Synthetic oligonucleotide
cttaaaattcgccttaatgccattcttttgcttatctgcggtaatgtatacattatgggaattgaaattatactccagcttatggccgag

SEQ ID NO: 63 - DNA, 90 bp, Artificial Sequence
  Description of Artificial Sequence Synthetic oligonucleotide
ctttttcttccgccagttgatcaaagtccagccagtcgttctattatcacattgtcagttatcatcttcggttacggttatcggcagaac

SEQ ID NO: 64 - DNA, 36 bp, Artificial Sequence
  Description of Artificial Sequence Synthetic oligonucleotide
ccgataaccgtaaccgaagatgataactgacaatgg

SEQ ID NO: 65 - DNA, 36 bp, Artificial Sequence
  Description of Artificial Sequence Synthetic oligonucleotide
ccgataaccgtaaccgaagatgataactgacaatgt

SEQ ID NO: 66 - DNA, 28 bp, Artificial Sequence
  Description of Artificial Sequence Synthetic oligonucleotide
cgtacttgttcaccatcgccactttggt

SEQ ID NO: 67 - DNA, 40 bp, Artificial Sequence
  Description of Artificial Sequence Synthetic oligonucleotide
cgactgagcccgaggagtaaaagcttgatgggggatccca

SEQ ID NO: 68 - DNA, 52 bp, Artificial Sequence
  Description of Artificial Sequence Synthetic oligonucleotide
tcaacaggactatcagacatggtatatctccttttattattaattcacatcc

SEQ ID NO: 69 - DNA, 43 bp, Artificial Sequence
  Description of Artificial Sequence Synthetic oligonucleotide
ataataaaaggagatataccatgtctgatagtcctgttgactt

SEQ ID NO: 70 - DNA, 40 bp, Artificial Sequence
  Description of Artificial Sequence Synthetic oligonucleotide
tgggatcccccatcaagcttttactcctcgggctcagtcg

SEQ ID NO: 71 - DNA, 39 bp, Artificial Sequence
  Description of Artificial Sequence Synthetic oligonucleotide
atgatgattacactccggcggagtttgttattgacgtgt

SEQ ID NO: 72 - DNA, 40 bp, Artificial Sequence
  Description of Artificial Sequence Synthetic oligonucleotide
cgtcaataacaaactccgccggagtgtaatcatcattgac

SEQ ID NO: 73 - DNA, 20 bp, Artificial Sequence
  Description of Artificial Sequence Synthetic oligonucleotide
taacctaaactgacaggcat

SEQ ID NO: 74 - DNA, 18 bp, Artificial Sequence
  Description of Artificial Sequence Synthetic oligonucleotide
ttccgatccccaattcct

SEQ ID NO: 75 - DNA, 19 bp, Artificial Sequence
  Description of Artificial Sequence Synthetic oligonucleotide
ggatcattttgcgcttcag

SEQ ID NO: 76 - DNA, 20 bp, Artificial Sequence
  Description of Artificial Sequence Synthetic oligonucleotide
gaatataacctttcattccc

SEQ ID NO: 77 - DNA, 19 bp, Artificial Sequence
  Description of Artificial Sequence Synthetic oligonucleotide
gtgtttcgactagcatttc

SEQ ID NO: 78 - DNA, 22 bp, Artificial Sequence
  Description of Artificial Sequence Synthetic oligonucleotide
ggtcaaacatgatttcaaaaac

SEQ ID NO: 79 - DNA, 18 bp, Artificial Sequence
  Description of Artificial Sequence Synthetic oligonucleotide
caacagtactgcgatgag

SEQ ID NO: 80 - DNA, 20 bp, Artificial Sequence
  Description of Artificial Sequence Synthetic oligonucleotide
gcaaataagctcttgtcagc

SEQ ID NO: 81 - DNA, 44 bp, Artificial Sequence
  Description of Artificial Sequence Synthetic oligonucleotide
  modified_base (21)..(21): a, c, t, g, unknown or other
catctatgtataaagtgatantcgtcaatgatgattacactccg

SEQ ID NO: 82 - DNA, 20 bp, Artificial Sequence
  Description of Artificial Sequence Synthetic oligonucleotide
tatcactttatacatagatg

SEQ ID NO: 83 - DNA, 44 bp, Artificial Sequence
  Description of Artificial Sequence Synthetic oligonucleotide
  modified_base (21)..(21): a, c, t, g, unknown or other
attacactccgatggagtttnttattgacgtgttacaaaaattc

SEQ ID NO: 84 - DNA, 20 bp, Artificial Sequence
  Description of Artificial Sequence Synthetic oligonucleotide
aaactccatcggagtgtaat

SEQ ID NO: 85 - DNA, 39 bp, Artificial Sequence
  Description of Artificial Sequence Synthetic oligonucleotide
  modified_base (21)..(21): a, c, t, g, unknown or other
caacgcaattgatgctcgctnttcactaccaggggaagg

SEQ ID NO: 86 - DNA, 20 bp, Artificial Sequence
  Description of Artificial Sequence Synthetic oligonucleotide
agcgagcatcaattgcgttg

SEQ ID NO: 87 - DNA, 43 bp, Artificial Sequence
  Description of Artificial Sequence Synthetic oligonucleotide
  modified_base (21)..(21): a, c, t, g, unknown or other
cgagggagaatgagcatccantcctgtgtacgctagaaaaagc

SEQ ID NO: 88 - DNA, 20 bp, Artificial Sequence
  Description of Artificial Sequence Synthetic oligonucleotide
tggatgctcattctccctcg

SEQ ID NO: 89 - DNA, 19 bp, Artificial Sequence
  Description of Artificial Sequence Synthetic oligonucleotide
gcggatttgtcctactcag

SEQ ID NO: 90 - DNA, 22 bp, Artificial Sequence
  Description of Artificial Sequence Synthetic oligonucleotide
gctaacaggaggaattagatct

SEQ ID NO: 91 - DNA, 25 bp, Artificial Sequence
  Description of Artificial Sequence Synthetic oligonucleotide
ttgataatctaacaaggattatggg

SEQ ID NO: 92 - DNA, 45 bp, Artificial Sequence
  Description of Artificial Sequence Synthetic oligonucleotide
cccataatccttgttagattatcaaaggcattttgctattaaggg

SEQ ID NO: 93 - DNA, 22 bp, Artificial Sequence
  Description of Artificial Sequence Synthetic oligonucleotide
agatctaattcctcctgttagc

SEQ ID NO: 94 - DNA, 19 bp, Artificial Sequence
  Description of Artificial Sequence Synthetic oligonucleotide
taactcgaggctgttttgg

SEQ ID NO: 95 - DNA, 24 bp, Artificial Sequence
  Description of Artificial Sequence Synthetic oligonucleotide
catatgtatatctccttgtgcatc

SEQ ID NO: 96 - DNA, 41 bp, Artificial Sequence
  Description of Artificial Sequence Synthetic oligonucleotide
gatgcacaaggagatatacatatggggagtgggtctttcat

SEQ ID NO: 97 - DNA, 37 bp, Artificial Sequence
  Description of Artificial Sequence Synthetic oligonucleotide
ccaaaacagcctcgagttaggctttttctagcgtaca

SEQ ID NO: 98 - DNA, 40 bp, Artificial Sequence
  Description of Artificial Sequence Synthetic oligonucleotide
acccgatcatgcaggttaacgttatgcactacgatggtgt

SEQ ID NO: 99 - DNA, 37 bp, Artificial Sequence
  Description of Artificial Sequence Synthetic oligonucleotide
tcaccaccgaatttttccggacctttgatggtcagcg

SEQ ID NO: 100 - DNA, 20 bp, Artificial Sequence
  Description of Artificial Sequence Synthetic oligonucleotide
ccggaaaaattcggtggtga

SEQ ID NO: 101 - DNA, 20 bp, Artificial Sequence
  Description of Artificial Sequence Synthetic oligonucleotide
gttaacctgcatgatcgggt

SEQ ID NO: 102 - DNA, 19 bp, Artificial Sequence
  Description of Artificial Sequence Synthetic oligonucleotide
acgctctcctgagtaggac

SEQ ID NO: 103 - DNA, 24 bp, Artificial Sequence
  Description of Artificial Sequence Synthetic oligonucleotide
tcaccgacaaacaacagataaaac

SEQ ID NO: 104 - DNA, 40 bp, Artificial Sequence
  Description of Artificial Sequence Synthetic oligonucleotide
tatctgttgtttgtcggtgaacgtctcattttcgccagat

SEQ ID NO: 105 - DNA, 40 bp, Artificial Sequence
  Description of Artificial Sequence Synthetic oligonucleotide
gtcctactcaggagagcgtagtgtcaactttatggctagc

SEQ ID NO: 106 - DNA, 17 bp, Artificial Sequence
  Description of Artificial Sequence Synthetic oligonucleotide
cgacctgaatggaagcc

SEQ ID NO: 107 - DNA, 17 bp, Artificial Sequence
  Description of Artificial Sequence Synthetic oligonucleotide
catacacggtgcctgac

SEQ ID NO: 108 - DNA, 46 bp, Artificial Sequence
  Description of Artificial Sequence Synthetic oligonucleotide
aacgcagtcaggcaccgtgtatggagaaaaaaatcactggatatac

SEQ ID NO: 109 - DNA, 37 bp, Artificial Sequence
  Description of Artificial Sequence Synthetic oligonucleotide
gccggcttccattcaggtcgaaaaaattacgccccgc

SEQ ID NO: 110 - DNA, 69 bp, Artificial Sequence
  Description of Artificial Sequence Synthetic oligonucleotide
  modified_base (28)..(29): a, c, t, g, unknown or other
  modified_base (34)..(35): a, c, t, g, unknown or other
  modified_base (43)..(44): a, c, t, g, unknown or other
caaaatgctggatttgatataattatannkttgnnkgatttannkgcctatttaaaccagaaaggagag

SEQ ID NO: 111 - DNA, 33 bp, Artificial Sequence
  Description of Artificial Sequence Synthetic oligonucleotide
tataattatatcaaatccagcattttgtaaatc

SEQ ID NO: 112 - DNA, 69 bp, Artificial Sequence
  Description of Artificial Sequence Synthetic oligonucleotide
  modified_base (26)..(27): a, c, t, g, unknown or other
  modified_base (29)..(30): a, c, t, g, unknown or other
  modified_base (44)..(45): a, c, t, g, unknown or other
ggcaaaatatgtttatggaagtgaannknnkcttgataaggatnnkacactgaatgtctatagattggc

SEQ ID NO: 113 - DNA, 25 bp, Artificial Sequence
  Description of Artificial Sequence Synthetic oligonucleotide
ttcacttccataaacatattttgcc

SEQ ID NO: 114 - DNA, 61 bp, Artificial Sequence
  Description of Artificial Sequence Synthetic oligonucleotide
  modified_base (22)..(23): a, c, t, g, unknown or other
  modified_base (28)..(29): a, c, t, g, unknown or other
  modified_base (40)..(41): a, c, t, g, unknown or other
gaagttatctatccaataatgnnkgttnnkggtgctcatnnkcttggcgttgatgttgcag

SEQ ID NO: 115 - DNA, 27 bp, Artificial Sequence
  Description of Artificial Sequence Synthetic oligonucleotide
cattattggatagataacttcagcaac

SEQ ID NO: 116 - DNA, 18 bp, Artificial Sequence
  Description of Artificial Sequence Synthetic oligonucleotide
cgcatcaggcaatttagc

SEQ ID NO: 117 - DNA, 43 bp, Artificial Sequence
  Description of Artificial Sequence Synthetic oligonucleotide
cgcgcgtgaagacgaaaaccagaaagttgcggaagttatctac

SEQ ID NO: 118 - DNA, 18 bp, Artificial Sequence
  Description of Artificial Sequence Synthetic oligonucleotide
ggttttcgtcttcacgcg

SEQ ID NO: 119 - DNA, 43 bp, Artificial Sequence
  Description of Artificial Sequence Synthetic oligonucleotide
tacccgatcatgcaggttaaaggtatccactacaaaggtgttg

SEQ ID NO: 120 - DNA, 20 bp, Artificial Sequence
  Description of Artificial Sequence Synthetic oligonucleotide
ttaacctgcatgatcgggta

SEQ ID NO: 121 - DNA, 38 bp, Artificial Sequence
  Description of Artificial Sequence Synthetic oligonucleotide
gtaaaatccacatgctggcgtgtgaactgctgccgaaa

SEQ ID NO: 122 - DNA, 19 bp, Artificial Sequence
  Description of Artificial Sequence Synthetic oligonucleotide
cgccagcatgtggatttta

SEQ ID NO: 123 - DNA, 41 bp, Artificial Sequence
  Description of Artificial Sequence Synthetic oligonucleotide
tcctggaatacccgctgaccttcaaacgtccggaaaaattc

SEQ ID NO: 124 - DNA, 18 bp, Artificial Sequence
  Description of Artificial Sequence Synthetic oligonucleotide
ggtcagcgggtattccag

SEQ ID NO: 125 - DNA, 40 bp, Artificial Sequence
  Description of Artificial Sequence Synthetic oligonucleotide
gctgaccatcaaacgtccggtaaaattcggtggtgacctg

SEQ ID NO: 126 - DNA, 17 bp, Artificial Sequence
  Description of Artificial Sequence Synthetic oligonucleotide
ccggacgtttgatggtc

SEQ ID NO: 127 - DNA, 39 bp, Artificial Sequence
  Description of Artificial Sequence Synthetic oligonucleotide
tcaaaaacaaagaactgcactcgatgcgtctgaaaaacg

SEQ ID NO: 128 - DNA, 22 bp, Artificial Sequence
  Description of Artificial Sequence Synthetic oligonucleotide
gtgcagttctttgtttttgaac

SEQ ID NO: 129 - DNA, 23 bp, Artificial Sequence
  Description of Artificial Sequence Synthetic oligonucleotide
ctgcagtttcaaacgctaaattg

SEQ ID NO: 130 - DNA, 50 bp, Artificial Sequence
  Description of Artificial Sequence Synthetic oligonucleotide
taggcctgataagcgtagcgcatcaggcaatttagcgtttgaaactgcag

SEQ ID NO: 131 - DNA, 39 bp, Artificial Sequence
  Description of Artificial Sequence Synthetic oligonucleotide
aatacccgctgaccatcaaacgtccggaaaaattcggtg

SEQ ID NO: 132 - DNA, 40 bp, Artificial Sequence
  Description of Artificial Sequence Synthetic oligonucleotide
accaccgaatttttccggacgtttgatggtcagcgggtat

SEQ ID NO: 133 - DNA, 20 bp, Artificial Sequence
  Description of Artificial Sequence Synthetic oligonucleotide
gcgaaatacgtttacggttc

SEQ ID NO: 134 - DNA, 20 bp, Artificial Sequence
  Description of Artificial Sequence Synthetic oligonucleotide
gaaccgtaaacgtatttcgc

SEQ ID NO: 135 - DNA, 21 bp, Artificial Sequence
  Description of Artificial Sequence Synthetic oligonucleotide
ggacggtgaaggtaaaatgtc

SEQ ID NO: 136 - DNA, 21 bp, Artificial Sequence
  Description of Artificial Sequence Synthetic oligonucleotide
gacattttaccttcaccgtcc

SEQ ID NO: 137 - DNA, 40 bp, Artificial Sequence
  Description of Artificial Sequence Synthetic oligonucleotide
cggcgccagggttgtttttcacgctctcctgagtaggaca

SEQ ID NO: 138 - DNA, 41 bp, Artificial Sequence
  Description of Artificial Sequence Synthetic oligonucleotide
ttccattcaggtcgaaaaaaagtgtcaactttatggctagc

SEQ ID NO: 139 - DNA, 22 bp, Artificial Sequence
  Description of Artificial Sequence Synthetic oligonucleotide
ttttttcgacctgaatggaagc

SEQ ID NO: 140 - DNA, 18 bp, Artificial Sequence
  Description of Artificial Sequence Synthetic oligonucleotide
gaaaaacaaccctggcgc

SEQ ID NO: 141 - DNA, 40 bp, Artificial Sequence; identical to SEQ ID NO: 137
  Description of Artificial Sequence Synthetic oligonucleotide

SEQ ID NO: 142 - DNA, 40 bp, Artificial Sequence
  Description of Artificial Sequence Synthetic oligonucleotide
ttccattcaggtcgaaaaaactcgaggtgaagacgaaagg

SEQ ID NO: 143 - DNA, 45 bp, Artificial Sequence
  Description of Artificial Sequence Synthetic oligonucleotide
acatttcagggaaggatgtgaattaataataaaaggagatatacc

SEQ ID NO: 144 - DNA, 31 bp, Artificial Sequence
  Description of Artificial Sequence Synthetic oligonucleotide
gcgtaccatgggatcccccatcaagctttta

SEQ ID NO: 145 - DNA, 20 bp, Artificial Sequence
  Description of Artificial Sequence Synthetic oligonucleotide
taaaagcttgatgggggatc

SEQ ID NO: 146 - DNA, 32 bp, Artificial Sequence
  Description of Artificial Sequence Synthetic oligonucleotide
ggtatatctccttttattattaattcacatcc

SEQ ID NO: 147 - DNA, 18 bp, Artificial Sequence
  Description of Artificial Sequence Synthetic oligonucleotide
ggatcatcgcgacatttc

SEQ ID NO: 148 - DNA, 19 bp, Artificial Sequence
  Description of Artificial Sequence Synthetic oligonucleotide
taataaaaggagatatacc

SEQ ID NO: 149 - protein, 106 aa, Escherichia coli
MGKTNDWLDFDQLAEEKVRDALKPPSMYKVILVNDDYTPMEFVIDVLQKFFSYDVERATQLMLAVHYQGKAICGVFTAEVAETKVAMVNKYARENEHPLLCTLEKA

SEQ ID NO: 150 - protein, 99 aa, Corynebacterium glutamicum
MSSPSAPLATPDVELDVHTLSSENLPWLCIVWDDPVNLMSYVTYVFQTVLGFSKKRATELMMQVHTEGKAVVSSGEKDKVEGDVKKLHTAGLWATMQQA

SEQ ID NO: 151 - protein, 94 aa, Streptomyces coelicolor
MEIEKTESAEEVFAVPEPDVPWVTIVHNDPVNLMSYVTYVFQSYFGYSKDKATKLMMDVHHKGRAVVSSGSREEMERDVQAMHGYGLWATLQQD

SEQ ID NO: 152 - protein, 106 aa, Vibrio cholerae
MSKNFEWISPDFDLLEKEKTAVKPPSMYHVVLNNDDYTPMDFVIEILERFFSMDIERATQVMLKVHYEGKAICGTFTAEVAETKVAQVTMYSRENEHPLLCTMEQA

SEQ ID NO: 153 - protein, 109 aa, Caulobacter crescentus
MAERKQGGQGNGVGSSVVTEVKPKTQKPSLYRVLILNDDYTPMEFVVYVLERFFNKSREDATRIMLHVHQNGVGVCGVYTYEVAETKVAQVIDSARRHQHPLQCTMEKD

SEQ ID NO: 154 - protein, 98 aa, Synechocystis sp.
MISNAATSTPDRLTSTVRKTYPNFKVIVLNDDFNTFQHVSDCLLKYIPGMTGDRAWELTNQVHFDGLAIVWVGPQEQAELYHQQLRREGLTMAPLEKA

SEQ ID NO: 155 - protein, 100 aa, Neisseria gonorrhoeae
MTAQHQSDTLLHRLNTLPPKRYGVFLLNDDYTTMEFVVEILTEIFMLGQEQAVAVMLSVHHEGKGLCGTYTRDIAQTKQQQVMQRAKAEGHPLQCIVEEI

SEQ ID NO: 156 - protein, 96 aa, Campylobacter jejuni
MPKTQTLEQTKLSEPKMYKVILLNDDVTTMDFVIEILMNIFHQNLEKASQTMLEIHHNGSGICGIYTQEIALSKQKKVMDAAKLANFPLQAKVEEE

SEQ ID NO: 157 - protein, 106 aa, Acinetobacter baylyi
MGDSQSDEPEGDIAVQTAPPELKRPPLYAVVLLNDDYTPMDFVIEILQQYFALNLDQATQVMLTVHYEGKGVAGVYPRDIAETKANQVNNYARSQGHPLLCQIEPK

SEQ ID NO: 158 - protein, 105 aa, Aeromonas salmonicida
MSKQKELFANEEIAQAEKTKLQPPPMYKVVLNNDDYTPMEFVVEVLQKFFGMDLDKATQVMLSVHYSGKGVCGTFTAEIAETKVVQVNTYSRNNEHPLLCTMEKA

SEQ ID NO: 159 - protein, 100 aa, Saccharomyces cerevisiae
DGTTTAKTSPSNSPEASPSLAKIDPENYTVIIYNDEYHNYSQATTALRQGVPDNVHIDLLTSRIDGEGRAMLKCSQDLSSVLGGFFAVQTNGLSATLTSW

SEQ ID NO: 160 - protein, 102 aa, Homo sapiens
MTIWEEEKELPPELQIREKNERYYCVLFNDEHHSYDHVIYSLQRALDCELAEAQLHTTAIDKEGRRAVKAGAYAACQEAKEDIKSHSENVSQHPLHVEVLHS

SEQ ID NO: 161 - protein, 6 aa, Artificial Sequence
  Description of Artificial Sequence Synthetic 6xHis tag
HHHHHH

SEQ ID NO: 162 - protein, 7 aa, Artificial Sequence
  Description of Artificial Sequence Synthetic peptide
  MOD_RES (1)..(1): Any amino acid
XLFVQEL

(19) United States
(12) Patent Application Publication
(10) Pub. No.: US 2025/0346939 A1
(43) Pub. Date: Nov. 13, 2025

(54) Method of Making Proteins with Non-Standard Amino Acids

(71) Applicant: President and Fellows of Harvard College, Cambridge, MA (US)

(72) Inventors: Aditya Mohan Kunjapur, Cambridge, MA (US); George M. Church, Brookline, MA (US)

(73) Assignee: President and Fellows of Harvard College, Cambridge, MA (US)

(21) Appl. No.: 16/484,869

(22) PCT Filed: Feb. 9, 2018
(86) PCT No.: PCT/US18/17581

§ 371 Date: Aug. 9, 2019

Related U.S. Application Data
(60) Provisional application No. 62/457,353, filed on Feb. 10, 2017.

(51) Int. Cl. C12P 21/06; C12N 9/00; C12N 9/52; C12N 15/62

(52) U.S. Cl. CPC C12P 21/06; C12N 9/52; C12N 9/93; C12N 15/62; C07K 2319/50; C07K 2319/60; C12Y 304/21; C12Y 601/01

(57) ABSTRACT

The disclosure provides methods of making a protein having a desired non-standard amino acid incorporated at its N-terminus in a cell and methods of screening for an amino acyl tRNA synthetase variant that preferentially selects a non-standard amino acid against its standard amino acid counterpart or undesired non-standard amino acids for incorporation into a protein in a cell.

### RELATED APPLICATION DATA

This application claims priority to U.S. Provisional Application No. 62/457,353 filed on Feb. 10, 2017 and U.S. Provisional Application No. 62/526,671 filed on Jun. 29, 2017, each of which is hereby incorporated herein by reference in its entirety for all purposes.

### STATEMENT OF GOVERNMENT INTERESTS

This invention was made with government support under DE-FG02-02ER63445 awarded by Department of Energy. The government has certain rights in the invention.

### SEQUENCE LISTING

The instant application contains a Sequence Listing which has been submitted electronically in ASCII format and is hereby incorporated by reference in its entirety. Said ASCII copy, created on Mar. 20, 2018, is named 010498_01053_WO_SL.txt and is 53,629 bytes in size.

### FIELD

The present invention relates in general to methods of making proteins with non-standard amino acids.

### BACKGROUND

Naturally-occurring (standard) amino acids (SAAs) are the 20 unique building blocks composing all proteins derived from biological systems. Non-standard amino acids (NSAAs) have been developed bearing functional groups beyond those encoded by the 20 standard amino acids. To date, more than 70 non-standard amino acids (NSAAs) have been developed for in vivo protein translation. See Liu et al., Annual Review of Biochemistry 79:413-444 (2010). Methods of incorporating NSAAs into proteins using engineered amino-acyl tRNA synthetases and transfer RNAs also inadvertently incorporate standard amino acids and non-target NSAAs due to promiscuity or nonspecificity of the engineered amino-acyl tRNA synthetases and transfer RNAs corresponding to the NSAAs. Recently, in vivo Escherichia coli orthogonal translation systems (OTSs) having engineered tRNA and aminoacyl-tRNA synthetase pairs that reassign the amber stop codon (UAG) were developed to characterize the performance of these orthogonal translation systems (OTSs) with respect to the efficiency and accuracy of NSAA incorporation. It was shown that in vivo incorporation of amino acids into reporter proteins (and thus reporter synthesis) at reassigned amber codons may not discriminate between standard and non-standard amino acids. See Monk J W, et al. (2016) Rapid and Inexpensive Evaluation of Nonstandard Amino Acid Incorporation in Escherichia coli. ACS Synthetic Biology. One method of determining the fidelity of NSAA incorporation includes in vitro protein purification followed by low-throughput mass spectrometry. Such an approach confirms the promiscuity of at least one E. coli OTS system (Methanococcus jannaschii tyrosyl-tRNA synthetase (MjTyrRS)-derived orthogonal synthetases). See Young T S, Ahmad I, Yin J A, & Schultz P G (2010) An Enhanced System for Unnatural Amino Acid Mutagenesis in E. coli. Journal of Molecular Biology 395(2):361-374. Misincorporation of a standard amino acid for a nonstandard amino acid may be detrimental in the production of antibodies containing NSAAs for conjugation or the production of biomaterials containing NSAAs for tunable properties. The incorporation of SAAs instead of NSAAs represents the formation of impurities and the heterogeneous mixture lowers yields. Improvements in synthetase and tRNA specificity or selectivity can improve rate of NSAA incorporation. However, analyzing the polypeptide for rate of NSAA incorporation is laborious making modification of the synthetase or tRNA in response to experimental results a slow process. See Santoro S W, Wang L, Herberich B, King D S, & Schultz P G (2002) An efficient system for the evolution of aminoacyl-tRNA synthetase specificity. Nat Biotech 20(10):1044-1048; and Amiram M, et al. (2015) Evolution of translation machinery in recoded bacteria enables multi-site incorporation of nonstandard amino acids. Nat Biotech 33(12):1272-1279.

There is a continuing need in the art to develop methods of making proteins with NSAAs with improved efficiency and accuracy and for modifying synthetases or tRNA to result in improved efficiency and accuracy.

### SUMMARY

The present disclosure provides a method for selectively degrading proteins having a standard amino acid at an amino acid target location within a mixture or collection or population of proteins including proteins having a nonstandard amino acid at the amino acid target location. In this context, the standard amino acid may be considered an undesired amino acid. The present disclosure also provides a method for selectively degrading proteins having an undesired non-target NSAA at an amino acid target location within a mixture or collection or population of proteins including proteins having a desired nonstandard amino acid at the amino acid target location. In this context, the undesired non-target NSAA may be considered an undesired amino acid. Aspects of the disclosure utilize methods and materials described herein to distinguish between different NSAAs at an amino acid target location when more than one NSAA is used during the synthesis procedure, such as when multiple different NSAAs are used simultaneously in vivo. According to one aspect, an in vivo methods are provided that can discriminate between NSAAs competing for incorporation at the same site of a protein.

According to one aspect, the methods may be carried out in vivo, i.e. within a cell. According to one aspect, methods of making target polypeptides having a non-standard amino acid substitution at an amino acid target location and methods of degrading target polypeptides having a standard amino acid substitution or an undesired NSAA (either referred to as an undesired amino acid) at the amino acid target location may be carried out in vivo, i.e. within a cell. According to one aspect, a target polypeptide is made which includes a non-standard amino acid at an amino acid target location using an engineered amino-acyl tRNA synthetase and a transfer RNA corresponding to the non-standard amino acid. A removable protecting group is attached to the target polypeptide adjacent to the amino acid target location, such that when the removable protecting group is removed, an N-end amino acid is exposed at the amino acid target location. Should the engineered amino-acyl tRNA synthetase and a transfer RNA add an undesired amino acid, i.e., a standard amino acid or an undesired NSAA, instead of the desired non-standard amino acid at the amino acid target location, methods are provided herein for degrading the target polypeptide including the undesired amino acid, i.e. a standard amino acid or an undesired NSAA, at the amino acid target location, such as by using the N-end rule pathway for protein degradation. According to one aspect, post-translational proofreading is used to distinguish correctly translated from incorrectly translated proteins. The disclosure provides a post-translational proofreading concept that results in protein degradation in a cell using the N-end rule unless the desired NSAA is incorporated at the N-terminus of the protein.

According to one aspect, the target polypeptide is made within a cell, i.e. in vivo, and the cell produces a protease and may also produce an adapter protein for the protease, i.e. a degradation system. Certain cells, such as E. coli, naturally produce the protease system ClpAP and the adaptor ClpS, and both components are present in order for the N-end rule degradation pathway to function. According to certain aspects, adaptor variants such as ClpS variants may be used which have different specificities than the natural ClpS. In this context, cell production of the natural ClpS be inactivated, for example, such as by gene modification where the ClpS gene sequence in the genome is modified to introduce premature stop codons. Such degradation systems are representative of a cellular system that degrades proteins according to the N-end rule pathway for protein degradation as is known in the art. According to the N-end rule, the half-life of a protein is determined by its N-terminal residue. An N-terminal residue is said to be destabilizing if it is recognized by a cellular degradation system, which in turn leads to degradation of the protein. If the N-terminal residue is not destabilizing, i.e, it is not recognized by a cellular degradation system, then the protein is not subject to the N-end rule pathway for protein degradation. Destabilizing amino acids may be natural amino acids. Destabilizing amino acids may be certain NSAAs. Destabilizing amino acids may be referred to as undesired amino acids. Stabilizing amino acids may be referred to as desired amino acids. Non-standard amino acids, such as analogs to natural amino acids, may be stabilizing or destabilizing amino acids. Certain non-standard amino acids, such as analogs to natural amino acids, may be resistant to degradation by cellular systems. In this manner, the method increases production (yield) of proteins having a desired amino acid, i.e., a stabilizing amino acid or an amino acid which is not destabilizing, such as certain non-standard amino acids, insofar as such a protein may not be subject to the N-end rule pathway for protein degradation. In this manner, the method decreases production (yield) of proteins having an undesired amino acid, such as a standard amino acid or a certain NSAA, insofar as such a protein may be subject to the N-end rule pathway for protein degradation. In this manner, the method determines successful desired NSAA incorporation to the extent that target polypeptides that include an undesired amino acid, such as a standard amino acid or a certain destabilizing NSAA (undesired NSAA), at the amino acid target location may be degraded and target polypeptides that include a desired non-standard amino acid at the amino acid target location remain and may not be degraded.

The present disclosure provides a method of optimizing production of proteins including a non-standard amino acid. Reaction conditions are provided for making a target polypeptide including a non-standard amino acid substitution at an amino acid target location using an engineered amino-acyl tRNA synthetase and a transfer RNA as is known in the art. A removable protecting group is attached to the target polypeptide adjacent to the amino acid target location, such that when the removable protecting group is removed, an N-end amino acid is exposed at the amino acid target location. Should the engineered amino-acyl tRNA synthetase and a transfer RNA add a standard amino acid or undesired NSAA instead of the desired non-standard amino acid at the amino acid target location, methods are provided herein for degrading the target polypeptide including the standard amino acid or undesired NSAA at the amino acid target location. The amount of proteins having a desired non-standard amino acid is determined. Given the amount of protein produced, the reaction conditions and/or the amino-acyl tRNA synthetase and/or tRNA are altered and the amount of proteins having the desired non-standard amino acid is again determined. The process is repeated until the process is optimized for a desired yield of protein including desired NSAA. Exemplary reaction conditions which may be altered according to the present disclosure include changes of culture media or concentration of desired NSAA. Alterations or changes to the amino-acyl tRNA synthetase and/or tRNA include one or more mutations that may improve performance of the amino-acyl tRNA synthetase and/or tRNA. Such mutations may be made by methods known to those of skill in the art such as random mutagenesis approaches such as error-prone polymerase chain reaction (PCR) or directed approaches such as site-saturation mutagenesis or rational point mutagenesis.

The present disclosure provides a method of distinguishing a protein having a nonstandard amino acid, i.e. a desired nonstandard amino acid, at an amino acid target location from a protein having a standard amino acid or undesired nonstandard amino acid at the amino acid target location by degrading the protein if a destabilizing amino acid (i.e., standard amino acid or undesired destabilizing NSAA) recognized by a protease is present at the amino acid target location.

The present disclosure provides a method of making a target protein using an amino-acyl tRNA synthetase engineered or designed to incorporate a target nonstandard amino acid (i.e., a desired NSAA) in the target protein at an amino acid target location, wherein a removable protecting group is adjacent the amino acid target location. The removable protecting group is removed to expose the amino acid target location in the presence of a degradation enzyme or degradation enzyme/adaptor complex. According to certain aspects, the adaptor discriminates between amino acids and accordingly whether degradation will occur by the degradation enzyme. According to one aspect, the degradation enzyme/adaptor complex degrades the target protein if a standard amino acid or undesired NSAA is present at the amino acid target location. If the desired nonstandard amino acid is present at the amino acid target location, then the degradation enzyme/adaptor complex is ineffective to degrade the target protein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The foregoing and other features and advantages of the present invention will be more fully understood from the following detailed description of illustrative embodiments taken in conjunction with the accompanying drawings in which:

FIGS. 1A and 1B illustrate methods described herein to selectively degrade proteins that contain one or more undesired amino acids at one or more target amino acid locations based on a N-end rule degradation strategy. FIG. 1A and FIG. 1B depict aspects of the present disclosure where a target protein including a standard amino acid at an amino acid target location is selectively degraded over the target protein including a non-standard amino acid at the amino acid target location. Degradation is carried out in vivo according to the N-end rule. FIG. 1A shows the three candidate site-specific OTS systems used previously in E. coli. FIG. 1B shows the components of the selective degradation methods using the N-end rule described herein. Figure discloses “*LFVQEL” as SEQ ID NO: 162. FIG. 1C shows measurements of in vivo fluorescence normalized to optical density (FL/OD) corresponding to different combinations of post-translational proofreading components. Expression of the orthogonal tRNA alone was not responsible for GFP synthesis in the absence of BipA, but expression of the BipARS/tRNA pair resulted in levels of FL/OD nearly as high in the absence of BipA as in the presence of BipA. Significant reduction of the FL/OD signal in the absence of BipA occurs upon expression of Ubp1, which cleaves the ubiquitin domain and exposes the amino acid corresponding to the first UAG codon as the N-terminal residue. The decrease in signal only in the absence of BipA suggests that BipARS was primarily mischarging tRNA with N-terminally destabilizing residues. The FL/OD signal in the absence of BipA further decreased upon expression of ClpS, suggesting that the rate of N-end discrimination was previously limiting. FIG. 1D shows a control experiment that demonstrates the expected effect of Ubp1/ClpS expression on FL/OD signal resulting from GFP protein with N-terminal UCG, UAC, or UAG codons. FIG. 1E shows the FL/OD as a function of the number of UAG codons.

FIGS. 2A and 2B shows the activities of different AARSs. FIG. 2A shows the activity of different amino-acyl tRNA synthetases (“AARS”) in the absence and presence of NSAA. FIG. 2B shows the activity of the AARSs when Ubp1 and ClpS are expressed. These results demonstrate that many tested AARSs mischarge tRNA in the absence of their intended NSAA, and that post-translational proofreading with wild-type ClpS can resolve differences for several of these AARS/NSAA pairs.

FIGS. 3A-3E shows the effect of various engineered ClpS variants and that ClpS variants may be engineered to be tunable to a particular substrate. FIG. 3A shows the activity profile of synthetase-1 on an NSAA panel to determine their natural N-end rule classification of these NSAAs. FIG. 3B shows the activity profile of synthetase-2 on structurally smaller NSAAs that were not substrates of synthetase-1. FIG. 3C shows the four ClpS residues within fourA of the para position of the N-terminal phenylalanine (F) on the substrate peptide. FIG. 3D shows the tunability achieved with rationally engineered ClpS variants. FIG. 3E shows further the properties of one ClpS variant of interest that leads to the classification of only two tested NSAAs as stabilizing.

FIGS. 4A-4B show sequence alignments of ClpS homologs. FIG. 4A shows the sequence alignments of 10 bacterial ClpS homologs including E. coli ClpS with the four ClpS residues of interest highlighted. Figure discloses SEQ ID NOS 149-158, respectively, in order of appearance. FIG. 4B shows the sequence alignments of E. coli ClpS together with portions of the yeast and human UBR1 E3 ubiquitin ligase protein homologs. According to certain aspects, the sequence alignments identify the region of UBR1 as suitable for rational engineering to adjust the N-end rule status of various NSAAs using methods known to those of skill in the art. Wild-type UBR1 in yeast and human cells contains an isoleucine (I) rather than valine (V) at the position equivalent to “ClpS_V65I”. Accordingly, one of skill will recognize that the natural N-end status of NSAAs may be different in eukaryotes than in prokaryotes. Figure discloses SEQ ID NOS 149, 159, and 160, respectively, in order of appearance.

### DETAILED DESCRIPTION

The present disclosure provides a method of making a target polypeptide in a cell, wherein the target polypeptide includes a desired non-standard amino acid substitution at an amino acid target location, i.e. the non-standard amino acid withstands degradation as described herein. As used herein, the terms “polypeptide” and “protein” include compounds that include amino acids joined together by peptide bonds between the carboxyl and amino groups of adjacent amino acid residues. Exemplary cells include prokaryotic cells and eukaryotic cells. Exemplary prokaryotic cells include bacteria, such as E. coli, such as genetically modified E. coli. The method includes genetically modifying the cell to express the target polypeptide including a desired non-standard amino acid substitution at an amino acid target location using an engineered amino-acyl tRNA synthetase and transfer RNA pair corresponding to the non-standard amino acid, and wherein the cell expresses the target polypeptide including a standard amino acid or an undesired NSAA at the amino acid target location when the engineered amino-acyl tRNA synthetase and transfer RNA pair non-selectively adds the standard amino acid or undesired NSAA at the amino acid target location. A removable protecting group is attached to the target polypeptide adjacent to the amino acid target location, such that when the removable protecting group is removed, an N-end amino acid is exposed at the amino acid target location. According to one aspect, the removable protecting group is orthogonal within the cell in which it is being used.

According to certain aspects, the cell includes a protease system for degrading the target polypeptide when the N-end amino acid is a standard amino acid. According to certain aspects, the cell includes a protease system for degrading the target polypeptide when the N-end amino acid is an undesired NSAA. According to certain aspects, the protease system includes an adapter protein and a corresponding protease. The adapter protein coordinates with the protease for degrading the target polypeptide when the N-end amino acid is a standard amino acid. According to one aspect, the protease system is endogenous. According to one aspect, the protease and adaptor can be expressed constitutively. According to one aspect, the protease system is exogenous. According to one aspect, the protease system is under influence of a promoter. According to one aspect, the adapter protein of the protease system is under influence of an inducible promoter. According to one aspect, the adapter protein is upregulated. According to one aspect, overexpression of adaptor to produce adaptor levels in excess of that found normally within a cell improves degradation of polypeptides having an undesired amino acid at the amino acid target location. According to one aspect, an adaptor protein is provided that facilitates N-end rule classification of an NSAA.

Because the N-end rule pathway of protein degradation is conserved across prokaryotes and eukaryotes, methods described herein are useful in prokaryotes and eukaryotes. The removable protecting groups should be orthogonal in the cell within which it is being used. Ubiquitin is a suitable protecting group in prokaryotic cells because it is orthogonal but it is not a suitable protecting group in eukaryotic cells because it is not orthogonal. In eukaryotic cells, ubiquitin is N-terminally added to proteins often to initiate the process of protein degradation in the proteasome. In addition, the adaptor proteins in eukaryotic cells are homologs of ClpS known as Ubiquitin E3 ligases. According to the present disclosure, ubiquitin E3 ligase domain is altered in order to change the N-end rule classification of an NSAA.

According to one aspect, the removable protecting group is removed to generate an N-end amino acid, and the protease degrades the target polypeptide when the N-end amino acid is a standard amino acid or an undesired NSAA. In this manner, the target polypeptide including a desired non-standard amino acid substitution, i.e. which is resistant to degradation, is enriched within the cell. According to one aspect, embodiments of the disclosure are directed to methods that allow selective degradation of proteins having a standard amino acid or undesired NSAA instead of a desired nonstandard amino acid at their N-termini in a cell. The methods can be used for producing proteins with desired nonstandard amino acids at their N-termini with no detectable impurities.

According to one aspect, a method of identifying the presence of a target polypeptide including a desired non-standard amino acid, i.e. one which is resistant to degradation, is provided. According to this aspect, the target polypeptide includes a detectable moiety attached to the C-end of the target polypeptide. In this manner, if the target polypeptide (and detectable moiety) that is made by the cell is not subject to degradation as described above, then the detectable moiety is detected as a measure of the amount of target polypeptide generated by the cell. Accordingly, a method is provided where a detectable moiety is present at the C-end of the target polypeptide, the removable protecting group is removed to generate an N-end amino acid, the protease (whether accompanied by an adapter protein or not depending upon the protease system being used) degrades the target polypeptide when the N-end amino acid is a standard amino acid or an undesired NSAA, for example, to thereby enrich the target polypeptide including a desired non-standard amino acid substitution, and the detectable moiety is detected as a measure of the amount of the target polypeptide including a desired non-standard amino acid substitution.

According to one aspect, a method is provided for engineering synthetases that are more selective for incorporating non-standard amino acids versus standard amino acids at a selected site in a protein. Since all or substantially all of proteins bearing a standard amino acid or an undesired NSAA at their N-terminus are degraded leaving only proteins with a desired nonstandard amino at their N-terminus, no or substantially no background signal due to standard amino acid or undesired NSAA incorporation results from the method. Synthetases can be evolved and their variants screened in a high-throughput fashion for their function of producing a protein incorporating a nonstandard amino acid, such as a desired NSAA. In this manner, those synthetases with improved function can be identified and modified further to further improve efficiency and selectivity.

I. Methods of Making a Target Polypeptide with an NSAA

In general, methods of making a target polypeptide that includes a non-standard amino acid are known. In general, a cell is genetically modified to include a nucleic acid sequence which encodes for the target polypeptide that includes one or more non-standard amino acids within its amino acid sequence. The cell can be genomically recoded, (“a genomically recoded organism”) to the extent that one or more codons have been reassigned to encode for a non-standard amino acid. For each different non-standard amino acid, an amino-acyl tRNA synthetase/tRNA pair is engineered and the cell is capable of using the amino-acyl tRNA synthetase/tRNA pair to add the corresponding non-standard amino acid (when present in the cell) to a growing peptide sequence. Materials, conditions, and reagents for genetically modifying a cell to make a target protein having one or more amino acid sequences are described in the following references, each of which are hereby incorporated by reference in their entireties.

Approaches to genomically recode organisms include multiplex automatable genome engineering (MAGE), (for example, as described in Wang, Harris H., et al. “Programming cells by multiplex genome engineering and accelerated evolution.” Nature 460.7257 (2009): 894-898 hereby incorporated by reference in its entirety) and hierarchical conjugative assembly genome engineering (CAGE) (for example, as described in Isaacs, Farren J., et al. “Precise manipulation of chromosomes in vivo enables genome-wide codon replacement.” Science 333.6040 (2011): 348-353 hereby incorporated by reference in its entirety). In addition, portions of recoded genomes can be synthesized and subsequently assembled, as described recently in an effort to construct a 57-codon organism (for example, as described in Ostrov, Nili, et al. “Design, synthesis, and testing toward a 57-codon genome.” Science 353.6301 (2016): 819-822 hereby incorporated by reference in its entirety). The modification of an organism, whether recoded or not recoded, in order to express a polypeptide containing a site-specific non-standard amino acid has been described extensively in the literature (for example, as described in Wang, Lei, et al. “Expanding the genetic code of Escherichia coli.” Science 292.5516 (2001): 498-500; Chin, Jason W., et al. “An expanded eukaryotic genetic code.” Science 301.5635 (2003): 964-967; Wang, Lei, and Peter G. Schultz. “Expanding the genetic code.” Angewandte chemie international edition 44.1 (2005): 34-66; Liu, Chang C., and Peter G. Schultz. “Adding new chemistries to the genetic code.” Annual review of biochemistry 79 (2010): 413-444; Chin, Jason W. “Expanding and reprogramming the genetic code of cells and animals.” Annual review of biochemistry 83 (2014): 379-408 each of which is hereby incorporated by reference in its entirety). In brief, foreign nucleic acid sequences containing a gene encoding an orthogonal amino-acyl tRNA synthetase and an associated tRNA are introduced into an organism, typically in an expression vector. In addition, a desired non-standard amino acid is added to the cell culture medium. A nucleic acid sequence corresponding to a target protein is modified so that a free codon, such as the UAG codon, is formed at the target site of the gene encoding the target protein. In the presence of these four components—aminoacyl tRNA synthetase protein, tRNA, NSAA, and target protein mRNA —the target protein containing the NSAA is made.

Basic to the present disclosure is the use of an amino-acyl tRNA synthetase/tRNA pair cognate to a nonstandard amino acid. Exemplary amino-acyl tRNA synthetase/tRNA pairs cognate to a nonstandard amino acid are known to those of skill in the art or may be designed for particular non-standard amino acids, as is known in the art or as described in Wang, Lei, and Peter G. Schultz. “Expanding the genetic code.” Angewandte chemie international edition 44.1 (2005): 34-66; Liu, Chang C., and Peter G. Schultz. “Adding new chemistries to the genetic code.” Annual review of biochemistry 79 (2010): 413-444; and Chin, Jason W. “Expanding and reprogramming the genetic code of cells and animals.” Annual review of biochemistry 83 (2014): 379-408 each of which are hereby incorporated by reference in its entirety.

According to one aspect, the amino-acyl tRNA synthetase/tRNA pair cognate to a nonstandard amino acid is orthogonal to the cellular components of the cell in which it is used. The orthogonality (and therefore the suitability) of exogenous amino-acyl tRNA synthetase/tRNA pairs is dependent on the type of host organism. Four main orthogonal aminoacyl-tRNA synthetases have been developed for genetic code expansion: the Methanococcus janaschii tyrosyl-tRNA synthetase (MjTyrRS)/tRNAcuA pair, the Escherichia coli tyrosyl-tRNA synthetase (EcTyrRS)/tRNAcuA pair, the E. coli leucyl-tRNA synthetase (EcLeuRS)/tRNACUA pair, and pyrrolysyl-tRNA synthetase (PylRS)/tRNAcuA pairs from certain Methanosarcina. The MjTyrRS/tRNAcuA pair is orthogonal in E. coli but not in eukaryotic cells. The EcTyrRS/tRNAcuA pair and the EcLeuRS/tRNAcuA pair are orthogonal in eukaryotic cells but not in E. coli, whereas the PylRS/tRNAcuA pair is orthogonal in bacteria, eukaryotic cells, and animals (see Chin, Jason W. “Expanding and reprogramming the genetic code of cells and animals.” Annual review of biochemistry 83 (2014): 379-408 hereby incorporated by reference in its entirety). To maintain orthogonality, the exogenous amino acyl tRNA synthetase should not recognize any native amino acids or native tRNA. To maintain orthogonality, the tRNA should not be recognized by any native amino-acyl tRNA synthetases. To maintain orthogonality, the non-standard amino acid should not be recognized by any native amino acyl tRNA synthetases. “Orthogonal” pairs meet one or more of the above conditions. It is to be understood that “orthogonal” pairs may lead to some mischarging, i.e. such as insubstantial mischarging for example, of orthogonal tRNA with native amino acids so long as sufficient efficiency of charging to the designed NSAA occurs.

Exemplary families of synthetases for bacteria in addition to those described above and incorporated by reference include the PylRS/tRNAcuA pair and the Saccharomyces cerevisiae tryptophanyl-tRNA synthetase (ScWRS)/tRNAcuA pair. These exemplary synthetase families have natural analogs (lysine and tryptophan) that are N-end destabilizing amino acids. The following references describe useful synthetase families and their associated NSAAs. Blight, Sherry K., et al. “Direct charging of tRNAcuA with pyrrolysine in vitro and in vivo.” Nature 431.7006 (2004): 333-335; Namy, Olivier, et al. “Adding pyrrolysine to the Escherichia coli genetic code.” FEBS letters 581.27 (2007): 5282-5288; Hughes, Randall A., and Andrew D. Ellington. “Rational design of an orthogonal tryptophanyl nonsense suppressor tRNA.” Nucleic acids research 38.19 (2010): 6813-6830; Ellefson, Jared W., et al. “Directed evolution of genetic parts and circuits by compartmentalized partnered replication.” Nature Biotechnology 32.1 (2014): 97-101; and Chatterjee, Abhishek, et al. “A Tryptophanyl-tRNA Synthetase/tRNA Pair for Unnatural Amino Acid Mutagenesis in E. coli.” Angewandte Chemie International Edition 52.19 (2013): 5106-5109 each of which are hereby incorporated by reference in its entirety. As is known in the art, the synthetase catalyzes a reaction that attaches the nonstandard amino acid to the correct tRNA. The amino-acyl tRNA then migrates to the ribosome. The ribosome adds the nonstandard amino acid where the tRNA anticodon corresponds to the reverse complement of the codon on the mRNA of the target protein to be translated.

### II. Removable Protecting Groups

According to one aspect, the target polypeptide includes a removable protecting group adjacent to the amino acid target location such that when the removable protecting group is removed, the amino acid target location is an N-end amino acid. Exemplary removable protecting groups are known to those of skill in the art and can be readily identified in the literature based on the present disclosure. According to one aspect, the removable protecting is a peptide sequence produced by the cell when making the target polypeptide. According to one aspect, the removable protecting is a peptide sequence produced by the cell when making the target polypeptide, such that the removable peptide and the target polypeptide is a fusion. According to this aspect, the cell is genetically modified to include a foreign nucleic acid sequence encoding the target polypeptide including a non-standard amino acid substitution at an amino acid target location and a removable protecting group attached to the target polypeptide adjacent to the amino acid target location. According to one aspect, the removable protecting group is foreign to the cell, i.e. it is not endogenous to the cell. In this manner, the removable protecting is orthogonal to endogenous enzymes or other conditions within the cell.

An exemplary removable protecting group includes a cleavable protecting group, such as an enzyme cleavable protecting group. According to one aspect, the cell produces an enzyme that cleaves the removable protecting group to generate an N-end amino acid. An exemplary removable protecting group is a protein that is cleavable by a corresponding enzyme. According to one aspect, a removable protecting group is foreign to the cell and is not endogenous. According to one aspect, the enzyme that cleaves the removable protecting group is foreign to the cell and is not endogenous. According to one aspect, an exemplary removable protecting group for prokaryotic cells is ubiquitin that is cleavable by Ubp1. According to another aspect, an exemplary removable protecting group for eukaryotic cells is the sequence MENLYFQ/*(SEQ ID NO: 1), where “*” is the target position for the NSAA (known in the field as the P1′ position), where “/” represents the cut site, and where “ENLYFQ/*” (SEQ ID NO: 2) is the sequence that is cleavable by certain variants of TEV protease. Ordinarily, TEV protease cleavage efficiency is influenced by the choice of the amino acid at the P1′ position. However, mutants of TEV protease have been engineered which have increased or altered substrate tolerance at the P1′ position (see Renicke, Christian, Roberta Spadaccini, and Christof Taxis. “A Tobacco Etch Virus Protease with Increased Substrate Tolerance at the P1′position.” PloS one 8.6 (2013): e67915 hereby incorporated by reference in its entirety). The use of TEV protease in vivo in mammalian cells has been demonstrated and is described in Oberst, Andrew, et al. “Inducible dimerization and inducible cleavage reveal a requirement for both processes in caspase-8 activation.” Journal of Biological Chemistry 285.22 (2010): 16632-16642 hereby incorporated by reference in its entirety. One of skill will readily understand based on the present disclosure that the methods described herein are useful in prokaryotic cells and eukaryotic cells.

According to the present disclosure, the N-end target residue is exposed using materials and methods that are or will become apparent to one of skill based on the present disclosure. An exemplary removable protecting protein domain includes a self-splicing domain, such as an intein, or other cleavable domains such as small ubiquitin modifiers (SUMO proteins). An exemplary removable protecting group may be a protein cleavage sequence along with its cognate partner, such as the TEV cleavage site and TEV protease. In general, any of the strategies used to remove N-terminal affinity tags in protein purification can serve as alternative ways to expose the N-end target residue. An exemplary system to expose the N-end target residue includes a class of enzymes known as methionine aminopeptidases which can remove the first N-terminal residue, such as when the second residue is the amino acid target location which is the desired site of addition of a NSAA. According to one aspect, the amino acid target location may be the N-terminal location or it may be any location between the N-terminal location and the C-terminal location. Accordingly, methods are provided for removing a protecting group and/or all amino acids up to the amino acid target location, thereby rendering the amino acid target location being the N-terminal amino acid.

### III. Detectable Moiety

According to one aspect, the target polypeptide includes a detectable moiety attached to the C-end of the target polypeptide. Exemplary detectable moieties are known to those of skill in the art and can be readily identified in the literature based on the present disclosure. According to one aspect, the detectable moiety is a peptide sequence produced by the cell when making the target polypeptide. According to one aspect, the detectable moiety is a peptide sequence produced by the cell when making the target polypeptide, such that the detectable moiety and the target polypeptide is a fusion. According to this aspect, the cell is genetically modified to include a foreign nucleic acid sequence encoding the target polypeptide including a non-standard amino acid substitution at an amino acid target location and a detectable moiety attached to the target polypeptide, for example, at the C-end of the target polypeptide. According to one aspect, the detectable moiety is foreign to the cell, i.e. it is not endogenous to the cell.

An exemplary detectable moiety is a fluorescent moiety, such as GFP, that can be detected by fluorimetry, for example. An exemplary detectable moiety is a reporter protein. An exemplary detectable moiety includes a protein that confers antibiotic resistance which can be detected in the presence of an antibiotic. An exemplary detectable moiety includes an enzyme that performs a function (such as Beta-Galactosidase) that can lead to easy colorimetric output.

Aspects of the methods described herein may make use of epitope tags and reporter gene sequences as detectable moieties. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-S-transferase (GST), horseradish peroxidase (HRP), chloramphenicol acetyltransferase (CAT) beta-galactosidase, betaglucuronidase, luciferase, green fluorescent protein (GFP), HcRed, DsRed, cyan fluorescent protein (CFP), yellow fluorescent protein (YFP), and autofluorescent proteins including blue fluorescent protein (BFP).

### IV. Genetic Modifications

Aspects of the present disclosure include the genetic modification of a cell to include foreign genetic material which can then be expressed by the cell. The cell may be modified to include any other genetic material or elements useful in the expression of a nucleic acid sequence. Foreign genetic elements may be introduced or provided to a cell using methods known to those of skill in the art. For example, the cell may be genetically modified to include a foreign nucleic acid sequence encoding the target polypeptide including a non-standard amino acid substitution at an amino acid target location, a removable protecting group attached to the target polypeptide adjacent to the amino acid target location and a detectable moiety attached to the C-end of the target polypeptide. The nonstandard amino acid may be encoded by a corresponding nonsense or sense codon. The cell may be genomically recoded to recognize an engineered amino-acyl tRNA synthetase corresponding or cognate to a non-standard amino acid. The cell may be genetically modified to include a foreign nucleic acid sequence encoding an amino-acyl tRNA synthetase and/or a transfer RNA corresponding or cognate to the nonstandard amino acid and wherein the nonstandard amino acid is provided to the cell and the cell expresses the synthetase and the transfer RNA to include the nonstandard amino acid at the amino acid target location. The cell is genetically modified to include a foreign nucleic acid sequence encoding an enzyme for cleaving the removable protecting group under influence of an inducible promoter. The cell is genetically modified to include an inducible promoter influencing the production of an enzyme system for removal of the removable protecting group. The enzyme system or component thereof may be under influence of the inducible promoter. For example, the adapter which helps associate the cleavage enzyme with the removable protecting group may be under influence of an inducible promoter.

In general, nucleic acids may be introduced into a cell using any method known to those skilled in the art for such introduction. Such methods include transfection, transduction, viral transduction, microinjection, lipofection, nucleofection, nanoparticle bombardment, transformation, conjugation and the like. One of skill in the art will readily understand and adapt such methods using readily identifiable literature sources.

Aspects of the methods described herein may make use of vectors. The term “vector” includes a nucleic acid molecule capable of transporting another nucleic acid to which it has been linked. Vectors used to deliver the nucleic acids to cells as described herein include vectors known to those of skill in the art and used for such purposes. Certain exemplary vectors may be plasmids, lentiviruses or adeno-associated viruses known to those of skill in the art. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, lentiviruses, replication defective retroviruses, adenoviruses, replication defective adenoviruses, and adeno-associated viruses). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” Common expression vectors of utility in recombinant DNA techniques are often in the form of plasmids. Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell).

Aspects of the methods described herein may make use of regulatory elements. The term “regulatory element” is intended to include promoters, enhancers, internal ribosomal entry sites (IRES), and other expression control elements (e.g. transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). Regulatory elements useful in eukaryotic cells include a tissue-specific promoter that may direct expression primarily in a desired tissue of interest, such as muscle, neuron, bone, skin, blood, specific organs (e.g. liver, pancreas), or particular cell types (e.g. lymphocytes). Regulatory elements may also direct expression in a temporal-dependent manner, such as in a cell-cycle dependent or developmental stage-dependent manner, which may or may not also be tissue or cell-type specific. In some embodiments, a vector may comprise one or more pol III promoter (e.g. 1, 2, 3, 4, 5, or more pol III promoters), one or more pol II promoters (e.g. 1, 2, 3, 4, 5, or more pol II promoters), one or more pol I promoters (e.g. 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1α promoter and Pol II promoters described herein. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981). It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including fusion proteins or peptides, encoded by nucleic acids as described herein (e.g., clustered regularly interspersed short palindromic repeats (CRISPR) transcripts, proteins, enzymes, mutant forms thereof, fusion proteins thereof, etc.). Common prokaryotic promoters include IPTG (isopropyl B-D-1-thiogalactopyranoside) inducible, anhydrotetracycline inducible, or arabinose inducible promoters. Such promoters express genes only in the presence of IPTG, anhydrotetracycline, or arabinose in the medium. An exemplary promoter for use in bacteria such as E. coli to express aminoacyl tRNA synthetase is an arabinose inducible promoter. An exemplary promoter for use in bacteria such as E. coli to express a reporter protein is an anhydrotetracycline inducible promoter.

Aspects of the methods described herein may make use of terminator sequences. A terminator sequence includes a section of nucleic acid sequence that marks the end of a gene or operon in genomic DNA during transcription. This sequence mediates transcriptional termination by providing signals in the newly synthesized mRNA that trigger processes which release the mRNA from the transcriptional complex. These processes include the direct interaction of the mRNA secondary structure with the complex and/or the indirect activities of recruited termination factors. Release of the transcriptional complex frees RNA polymerase and related transcriptional machinery to begin transcription of new mRNAs. Terminator sequences include those known in the art and identified and described herein.

### V. Adapter Protein Protease Systems

According to one aspect, the cell includes a protease system for degrading the target polypeptide when the N-end amino acid is a standard amino acid. The protease system may be endogenous or exogenous. The cell may include an adapter or discriminator protein that coordinates with a protease for degrading the target polypeptide when the N-end amino acid is a standard amino acid. The adapter protein may be under influence of an inducible promoter. According to one aspect, the adapter protein is ClpS or a variant or mutant thereof. According to one aspect, adapter proteins may have different levels of selectivity for certain amino acids. According to certain aspects, adapter proteins, such as ClpS may be altered to improve selectivity, such as between standard amino acids and non-standard amino acids or between a desired NSAA and an undesired NSAA. According to one aspect, the protease system is a ClpS-ClpAP protease system.

According to one aspect, protease systems include Clps or homologs or mutants thereof, such as ClpS_V65I. The N-end rule is mediated by homologs of ClpS/ClpAP in bacteria. In eukaryotes, the N-end rule involves more distant homologs of ClpS (UBR1, ubiquitin E3 ligases) and degradation by the proteasome. Accordingly, the present disclosure contemplates use of many of the bacterial ClpS homologs to perform similar functions with slightly different amino acid recognition specificity. The present disclosure also contemplates use of eukaryotic protease systems, such as UBR1 and related variants to mediate N-end rule recognition with different amino acid recognition specificity in eukaryotes.

### VI. Cells

According to certain aspects, cells according to the present disclosure include prokaryotic cells and eukaryotic cells. Exemplary prokaryotic cells include bacteria. Microorganisms which may serve as host cells and which may be genetically modified to produce recombinant microorganisms as described herein may include one or members of the genera Clostridium, Escherichia, Rhodococcus, Pseudomonas, Bacillus, Lactobacillus Saccharomyces, and Enterococcus. Particularly suitable microorganisms include bacteria and archaea. Exemplary microorganisms include Escherichia coli, Bacillus subtilis, and Saccharomyces cerevisiae. Exemplary eukaryotic cells include animal cells, such as human cells, plant cells, fungal cells and the like.

In addition to E. coli, other useful bacteria include but are not limited to Bacillus subtilis, Bacillus megaterium, Bifidobacterium bifidum, Caulobacter crescentus, Clostridium difficile, Chlamydia trachomatis, Corynebacterium glutamicum, Lactobacillus acidophilus, Lactococcus lactis, Mycoplasma genitalium, Neisseria gonorrhoeae, Prochlorococcus marinus, Pseudomonas aeruginosa, Psuedomonas putida, Treponema pallidum, Streptomyces coelicolor, Synechococcus elongates, Vibrio natrigiens, and Zymomonas mobilis.

Exemplary genus and species of bacteria cells include Acetobacter aurantius, Acinetobacter bitumen, Actinomyces israelii, Agrobacterium radiobacter, Agrobacterium tumefaciens, Anaplasma Anaplasma phagocytophilum, Azorhizobium caulinodans, Azotobacter vinelandii, viridans streptococci, Bacillus anthracis, Bacillus brevis, Bacillus cereus, Bacillus fusiformis, Bacillus licheniformis, Bacillus megaterium, Bacillus mycoides, Bacillus stearothermophilus, Bacillus subtilis, Bacteroides, Bacteroides fragilis, Bacteroides gingivalis, Bacteroides melaninogenicus (also referred to as Prevotella melaninogenica), Bartonella,Bartonella henselae, Bartonella quintana, Bordetella, Bordetella bronchiseptica, Bordetella pertussis, Borrelia burgdorferi, Brucella abortus, Brucella melitensis, Brucella suis, Burkholderia, Burkholderia mallei, Burkholderia pseudomallei, Burkholderia cepacia, Calymmatobacterium granulomatis, Campylobacter, Campylobacter coli, Campylobacter fetus, Campylobacter jejuni, Campylobacter pylori, Chlamydia, Chlamydia trachomatis, Chlamydophila Chlamydophila pneumoniae (also known as Chlamydia pneumoniae) Chlamydophila psittaci (also known as Chlamydia psittaci), Clostridium, Clostridium botulinum, Clostridium difficile, Clostridium perfringens (also known as Clostridium welchii), Clostridium tetani, Corynebacterium, Corynebacterium diphtheriae, Corynebacterium fusiforme, Coxiella burnetii, Ehrlichia chaffeensis, Enterobacter cloacae, Enterococcus, Enterococcus avium, Enterococcus durans, Enterococcus faecalis, Enterococcus faecium, Enterococcus galllinarum, Enterococcus maloratus, Escherichia coli, Francisella tularensis, Fusobacterium nucleatum, Gardnerella vaginalis, Haemophilus, Haemophilus ducreyi, Haemophilus influenzae, Haemophilus parainfluenzae, Haemophilus pertussis, Haemophilus vaginalis, Helicobacter pylori, Klebsiella pneumoniae, Lactobacillus, Lactobacillus acidophilus, Lactobacillus bulgaricus, Lactobacillus casei, Lactococcus lactis, Legionella pneumophila, Listeria monocytogenes, Methanobacterium extroquens, Microbacterium multiforme, Micrococcus luteus, Moraxella catarrhalis, Mycobacterium, Mycobacterium avium, Mycobacterium bovis, Mycobacterium diphtheriae, Mycobacterium intracellulare, Mycobacterium leprae, Mycobacterium lepraemurium, Mycobacterium phlei, Mycobacterium smegmatis, Mycobacterium tuberculosis, Mycoplasma, Mycoplasma fermentans, Mycoplasma genitalium, Mycoplasma hominis, Mycoplasma penetrans, Mycoplasma pneumoniae, Neisseria, Neisseria gonorrhoeae, Neisseria meningitidis, Pasteurella, Pasteurella multocida, Pasteurella tularensis, Peptostreptococcus, Porphyromonas gingivalis, Prevotella melaninogenica (also known as Bacteroides melaninogenicus), Pseudomonas aeruginosa, Rhizobium radiobacter, Rickettsia, Rickettsia prowazekii, Rickettsia psittaci, Rickettsia quintana, Rickettsia rickettsii, Rickettsia trachomae, Rochalimaea, Rochalimaea henselae, Rochalimaea quintana, Rothia dentocariosa, Salmonella, Salmonella enteritidis, Salmonella typhi, Salmonella typhimurium, Serratia marcescens, Shigella dysenteriae, Staphylococcus, Staphylococcus aureus, Staphylococcus epidermidis, Stenotrophomonas maltophilia, Streptococcus Streptococcus agalactiae, Streptococcus avium, Streptococcus bovis, Streptococcus cricetus, Streptococcus faceium, Streptococcus faecalis, Streptococcus ferns, Streptococcus gallinarum, Streptococcus lactis, Streptococcus mitior, Streptococcus mitis, Streptococcus mutans, Streptococcus oralis, Streptococcus pneumoniae, Streptococcus pyogenes, Streptococcus rattus, Streptococcus salivarius, Streptococcus sanguis, Streptococcus sobrinus, Treponema, Treponema pallidum, Treponema denticola, Vibrio, Vibrio cholerae, Vibrio comma, Vibrio parahaemolyticus, Vibrio vulnificus, Wolbachia, Yersinia, Yersinia enterocolitica, Yersinia pestis, and Yersinia pseudotuberculosis, and other genus and species known to those of skill in the art.

Exemplary genus and species of yeast cells include Saccharomyces, Saccharomyces cerevisiae, Torula, Saccharomyces boulardii, Schizosaccharomyces, Schizosaccharomyces pombe, Candida, Candida glabrata, Candida tropicalis, Yarrowia, Candida parapsilosis, Candida krusei, Saccharomyces pastorianus, Brettanomyces, Brettanomyces bruxellensis, Pichia, Pichia guilliermondii, Cryptococcus, Cryptococcus gattii, Torulaspora, Torulaspora delbrueckii, Zygosaccharomyces, Zygosaccharomyces bailii, Candida lusitaniae, Candida stellata, Geotrichum, Geotrichum candidum, Pichia pastoris, Kluyveromyces, Kluyveromyces marxianus, Candida dubliniensis, Kluyveromyces, Kluyveromyces lactis, Trichosporon, Trichosporon uvarum, Eremothecium, Eremothecium gossypii, Pichia stipitis, Candida milleri, Ogataea, Ogataea polymorpha, Candida oleophilia, Zygosaccharomyces rouxii, Candida albicans, Leucosporidium, Leucosporidium frigidum, Candida viswanathii, Candida blankii, Saccharaomyces telluris, Saccharomyces florentinus, Sporidiobolus, Sporidiobolus salmonicolor, Dekkera, Dekkera anomala, Lachancea, Lachancea kluyveri, Trichosporon, Trichosporon mycotoxinivorans, Rhodotorula, Rhodotorula rubra, Saccharomyces exiguus, Sporobolomyces koalae, and Trichosporon cutaneum, and other genus and species known to those of skill in the art.

Exemplary genus and species of fungal cells include Sac fungi, Basidiomycota, Zygomycota, Chtridiomycota, Basidiomycetes, Hyphomycetes, Glomeromycota, Microsporidia, Blastocladiomycota, and Neocallimastigomycota, and other genus and species known to those of skill in the art.

Exemplary eukaryotic cells include mammalian cells, plant cells, yeast cells and fungal cells.

### VII. Standard Amino Acid

As used herein, the term “SAA” (standard amino acid) include one of the L-amino acids that typically naturally occur in proteins on Earth and includes alanine, arginine, asparagine, aspartic acid, cysteine, glutamic acid, glutamine, glycine, histidine, isoleucine, leucine, lysine, methionine, phenylalanine, serine, threonine, tyrosine, tryptophan, proline and valine. The standard amino acids that are naturally N-end destabilizing in most bacteria include tyrosine, phenylalanine, tryptophan, leucine, lysine, and arginine. According to one aspect, the amino acid at the amino acid target location is an NSAA that is stabilizing. When the natural analog of the NSAA is destabilizing and is present at the amino acid target location, degradation of the polypeptide occurs. Standard amino acids that are not naturally destabilizing via the N-end rule using natural ClpS, can be destabilizing when the ClpS is engineered to recognize such standard amino acid.

The N-end rule in bacteria may also be engineered to recognize isoleucine, valine, aspartate, glutamate, asparagine, and glutamine as destabilizing using methods known to those of skill in the art which is useful when the desired NSAA is an analog of these amino acids. For example, isoleucine and valine can be converted into N-end destabilizing residues by introducing a ClpS variant (M40A) that recognizes these amino acids as N-terminal destabilizing residues see (Román-Hernández G, Grant R A, Sauer R T, & Baker T A (2009) Molecular basis of substrate selection by the N-end rule adaptor protein ClpS. Proceedings of the National Academy of Sciences 106(22):8888-8893 hereby incorporated by reference in its entirety). Aspartate and glumatate may be converted into N-end destabilizing residues by introducing a bacterial aminoacyl-transferase from Vibrio vulificus (Bpt) that is a homolog of eukaryotic transferases and N-terminally appends a leucine (L) to peptides containing N-terminally exposed aspartate or glutamate (see Graciet E, et al. (2006) Aminoacyl-transferases and the N-end rule pathway of prokaryotic/eukaryotic specificity in a human pathogen. Proceedings of the National Academy of Sciences of the United States of America 103(9):3078-3083 hereby incorporated by reference in its entirety). The ability of Bpt to catalyze this reaction has been demonstrated in E. coli and shows that components of the N-end rule, which includes many more conditionally destabilizing residues in eukaryotes, can be transferred across kingdoms. Asparagine and glutamine can be converted into N-end destabilizing residues by using an N-terminal amidase from S. cerevisiae (NTA1), which converts N-terminal asparagine into aspartate or N-terminal glutamine into glumate, respectively (see Tasaki T, Sriram S M, Park K S, & Kwon Y T (2012) The N-End Rule Pathway. Annual Review of Biochemistry 81(1):261-289 hereby incorporated by reference in its entirety). Indeed, in many eukaryotic cells these amino acids and more are naturally conditionally N-end destabilizing. One of skill will understand that an N-end rule destabilizing pathway may be provided for all 20 standard amino acids as a basis for a system where a desired amino acid from among the 20 standard amino acids is N-end destabilizing in at least one context (see Chen, Shun-Jia, et al. “An N-end rule pathway that recognizes proline and destroys gluconeogenic enzymes.” Science 355.6323 (2017): eaal3655 hereby incorporated by reference in its entirety). One of skill in the art can identify the eukaryotic proteins required for conferring expanded N-end destabilization and transfer them to prokaryotes as needed. Similarly, in eukaryotic cells one can constitutively express components required for conferring expanded N-end destabilization such that degradation of proteins containing N-end standard amino acids no longer remains conditional. One of skill will recognize that some amino acids rendered destabilizing may have adverse consequences for cell physiology. For example, most native proteins begin with methionine and if methionine is made N-end destabilizing then most proteins would degrade. Aspects of converting an N-end stabilizing amino acid to an N-end destabilizing amino acid can be tested in a particular organism.

### VIII. Non-Standard Amino Acid

As used herein, the term “NSAA” refers to an unmodified amino acid that is not one of the 20 naturally occurring standard L-amino acids. NSAAs also include synthetic amino acids which have been designed to include a non-standard functional group not present in the standard amino acids or are naturally occurring amino acids bearing functional groups not present in the set of standard amino acids. Accordingly, a non-standard amino acid may include the structure of a standard amino acid and which includes a non-standard functional group. A non-standard amino acid may include the basic amino acid portion of a standard amino acid and include a non-standard functional group.

NSAAs also refer to natural amino acids that are not used by all organisms (e.g. L-pyrrolysine (B. Hao et al., A new uag-encoded residue in the structure of a methanogen methyltransferase. Science. 296:1462) and L-selenocysteine (S. Osawa et al., Recent evidence for evolution of the genetic code. Microbiol. Mol. Biol. Rev. 56:229)). NSAAs are also known in the art as unnatural amino acids (UAAs) and non-canonical amino acids (NCAAs).

NSAAs include, but are not limited to, p-Acetylphenylalanine, m-Acetylphenylalanine, O-allyltyrosine, Phenylselenocysteine, p-Propargyloxyphenylalanine, p-Azidophenylalanine, p-Boronophenylalanine, O-methyltyrosine, p-Aminophenylalanine, p-Cyanophenylalanine, m-Cyanophenylalanine, p-Fluorophenylalanine, p-Iodophenylalanine, p-Bromophenylalanine, p-Nitrophenylalanine, L-DOPA, 3-Aminotyrosine, 3-Iodotyrosine, p-Isopropylphenylalanine, 3-(2-Naphthyl)alanine, biphenylalanine, homoglutamine, D-tyrosine, p-Hydroxyphenyllactic acid, 2-Aminocaprylic acid, bipyridylalanine, HQ-alanine, p-Benzoylphenylalanine, o-Nitrobenzylcysteine, o-Nitrobenzylserine, 4,5-Dimethoxy-2-Nitrobenzylserine, o-Nitrobenzyllysine, o-Nitrobenzyltyrosine, 2-Nitrophenylalanine, dansylalanine, p-Carboxymethylphenylalanine, 3-Nitrotyrosine, sulfotyrosine, acetyllysine, methylhistidine, 2-Aminononanoic acid, 2-Aminodecanoic acid, pyrrolysine, Cbz-lysine, Boc-lysine, allyloxycarbonyllysine, arginosuccinic acid, citrulline, cysteine sulfinic acid, 3,4-dihydroxyphenylalanine, homocysteine, homoserine, ornithine, 3-monoiodotyrosine, 3,5-diiodotryosine, 3,5,5,-triiodothyronine, and 3,3′,5,5′-tetraiodothyronine. Modified or unusual amino acids include D-amino acids, hydroxylysine, 4-hydroxyproline, N-Cbz-protected amino acids, 2,4-diaminobutyric acid, homoarginine, norleucine, N-methylaminobutyric acid, naphthylalanine, phenylglycine, -phenylproline, tert-leucine, 4-aminocyclohexylalanine, N-methyl-norleucine, 3,4-dehydroproline, N,N-dimethylaminoglycine, N-methylaminoglycine, 4-aminopiperidine-4-carboxylic acid, 6-aminocaproic acid, trans-4-(aminomethyl)-cyclohexanecarboxylic acid, 2-, 3-, and 4-(aminomethyl)-benzoic acid, 1-aminocyclopentanecarboxylic acid, 1-aminocyclopropanecarboxylic acid, and 2-benzyl-5-aminopentanoic acid, and the like. NSAAs also include amino acids that are functionalized, e.g., alkyne-functionalized, azide-functionalized, ketone-functionalized, aminooxy-functionalized and the like. For reviews of NSAAs and lists of NSAAs suitable for use in certain embodiments of the subject invention, see Liu and Schultz (2010) Ann. Rev. Biochem. 79:413, and Kim et al. (2013) Curr. Opin. Chem. Biol. 17:412, each of which is incorporated herein by reference in its entirety for all purposes.

In certain aspects, an NSAA of the subject invention has a corresponding aminoacyl tRNA synthetase (aaRS)/tRNA pair. In certain aspects, the aminoacyl tRNA synthetase/tRNA pair is orthogonal to those in a genetically modified organism such as, e.g., a prokaryotic cell, a bacterium (e.g., E. coli), a eukaryotic cell, a yeast, a plant cell, an insect cell, a mammalian cell, a virus, etc. In certain aspects, an NSAA of the subject invention is non-toxic when expressed in a genetically modified organism such as, e.g., a prokaryotic cell, a bacterium (e.g., E. coli), a eukaryotic cell, a yeast, a plant cell, an insect cell, a mammalian cell, a virus, etc. In certain aspects, an NSAA of the subject invention is not or does not resemble a natural product present in a cell or organism. In certain aspects, an NSAA of the subject invention is hydrophobic, hydrophilic, polar, positively charged, or negatively charged. In other aspects, an NSAA of the subject invention is commercially available (such as, e.g., L-4,4-bipnehylalanine (bipA) and L-2-Naphthylalanine (napA)) or synthesized according to published protocols.

### EXAMPLE I

### Exemplary Degradation Materials and Methods

According to one aspect, the disclosure provides a method of making a protein having a non-standard amino acid incorporated therein, such as at its N-terminus, in a cell. The cell is provided with a nucleic acid sequence encoding a ubiquitin fused to the N-terminus of the protein wherein the N-terminus of the protein is an amino acid target location intended to have a nonstandard amino acid. The nonstandard amino acid may be encoded by a nonsense or sense codon. The cell is provided with a ubiquitin cleavase. The cell may include an endogenous protease system, such as a ClpS-ClpAP system. The cell is provided with a non-standard amino acid. The cell expresses the fusion protein having either a standard or a non-standard amino acid incorporated at the amino acid target location. The ubiquitin cleavase cleaves the ubiquitin to produce a protein having either the standard or non-standard intervening amino acid at its N-terminus. If a standard amino acid is present at the N-terminus, the ClpS recognizes the standard amino acid at the N-terminus and targets the protein having the standard amino acid at its N-terminus to ClpP for degradation. If a nonstandard amino acid is present at the N-terminus, the Clps does not recognize the nonstandard amino acid and the protein is not targeted for degradation. A residue is destabilizing if it is recognized by the ClpS adaptor protein, which is the discriminator of the N-end rule in E. coli such as is described in Erbse A, et al. (2006) ClpS is an essential component of the N-end rule pathway in Escherichia coli. Nature 439(7077):753-756 and Wang K H, Oakes ESC, Sauer R T, & Baker T A (2008) Tuning the Strength of a Bacterial N-end Rule Degradation Signal. Journal of Biological Chemistry 283(36):24600-24607; Schmidt R, Zahn R, Bukau B, & Mogk A (2009) ClpS is the recognition component for Escherichia coli substrates of the N-end rule degradation pathway. Molecular Microbiology 72(2):506-517; Romin-Hernindez G, Grant R A, Sauer R T, & Baker T A (2009) Molecular basis of substrate selection by the N-end rule adaptor protein ClpS. Proceedings of the National Academy of Sciences 106(22):8888-8893; Schuenemann V J, et al. (2009) Structural basis of N-end rule substrate recognition in Escherichia coli by the ClpAP adaptor protein ClpS. EMBO reports 10(5):508-514; Romin-Hernindez G, Hou Jennifer Y, Grant Robert A, Sauer Robert T, & Baker Tania A (2011) The ClpS Adaptor Mediates Staged Delivery of N-End Rule Substrates to the AAA+ClpAP Protease. Molecular Cell 43(2):217-228; and Hou J Y, Sauer R T, & Baker T A (2008) Distinct structural elements of the adaptor ClpS are required for regulating degradation by ClpAP. Nat Struct Mol Biol 15(3):288-294 each of which is hereby incorporated by reference in its entirety.

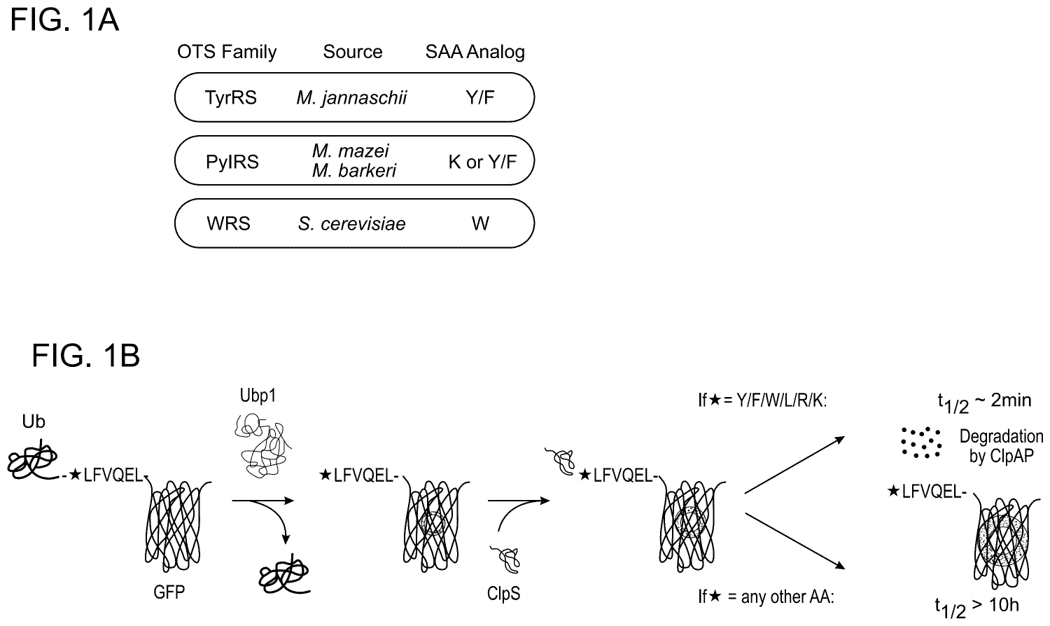

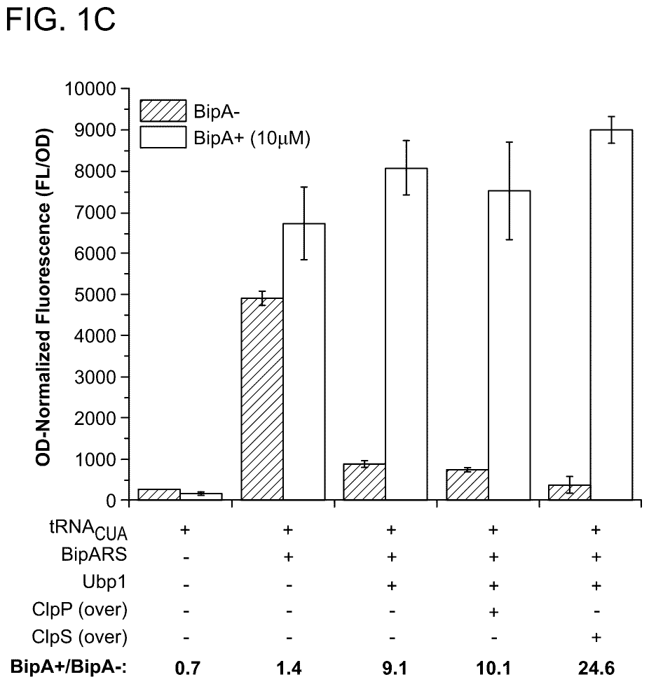

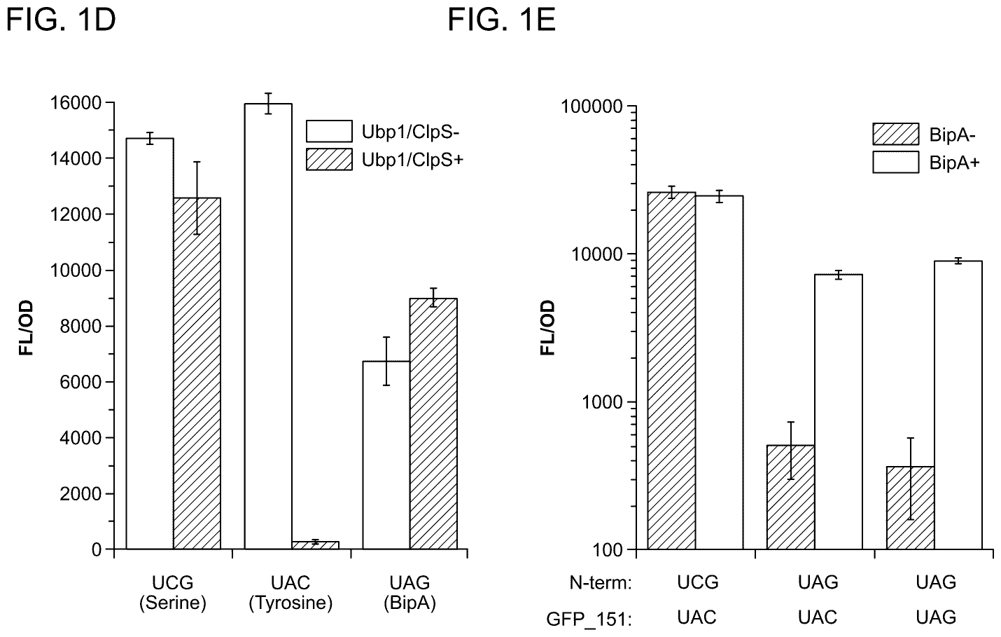

According to another aspect, the disclosure provides a method of screening for an amino acyl tRNA synthetase variant that preferentially selects a non-standard amino acid against its standard amino acid counterpart for incorporation into a protein in a cell. The cell is provided with an amino acyl tRNA synthetase variant. As shown in FIGS. 1A and 1B, the cell is provided with a nucleic acid sequence encoding a ubiquitin fused to the N-terminus of the protein wherein the N-terminus of the protein is an amino acid target location intended to have a nonstandard amino acid, and wherein GFP is fused to the C-end of the protein. The nonstandard amino acid may be encoded by a nonsense or sense codon. The cell is provided with a ubiquitin cleavase, such as Ubp1. The cell may include an endogenous protease system, such as a ClpS-ClpAP system. The cell is provided with a non-standard amino acid. The cell expresses the fusion protein having either a standard or a non-standard amino acid incorporated at the amino acid target location. The ubiquitin cleavase cleaves the ubiquitin to produce a protein having either the standard or non-standard intervening amino acid at its N-terminus. If a standard amino acid is present at the N-terminus, the ClpS recognizes the standard amino acid at the N-terminus and targets the protein having the standard amino acid at its N-terminus to ClpP for degradation, including the GFP portion. If a nonstandard amino acid is present at the N-terminus, the Clps does not recognize the nonstandard amino acid and the protein is not targeted for degradation. The GFP is detected and is indicative of the presence of a synthetase variant that preferentially selects the non-standard amino acid against its standard amino acid counterpart for incorporation into the protein.

According to another aspect, the strength of the signal detected from the GFP is indicative of the amount of protein produced that included the nonstandard amino acid. In this manner, methods are provided for screening and evolving an amino acyl tRNA synthetase variant that preferentially selects a non-standard amino acid against its standard amino acid counterpart for incorporation into a protein in a cell.

### EXAMPLE II

a Method of Making a Protein Having a Non-Standard Amino Acid Incorporated at its N-Terminus in an Engineered E. coli Having Orthogonal Translation Systems

To evaluate and classify various orthogonal translation systems in engineered E. coli for NSAA incorporation and proofreading, a fusion protein was constructed having a ubiquitin fused to the N-terminus of a GFP reporter protein having an intervening amino acid encoded by a UAG codon in between the ubiquitin and GFP. The plasmid containing the nucleic acid sequence of the fusion protein was introduced into the E. coli. In addition, the expression of a ubiquitin cleavase from yeast (Ubp1) was induced in E. coli as described in Tobias J W & Varshavsky A (1991) Cloning and functional analysis of the ubiquitin-specific protease gene UBP1 of Saccharomyces cerevisiae. J Biol Chem 266 hereby incorporated by reference in its entirety.

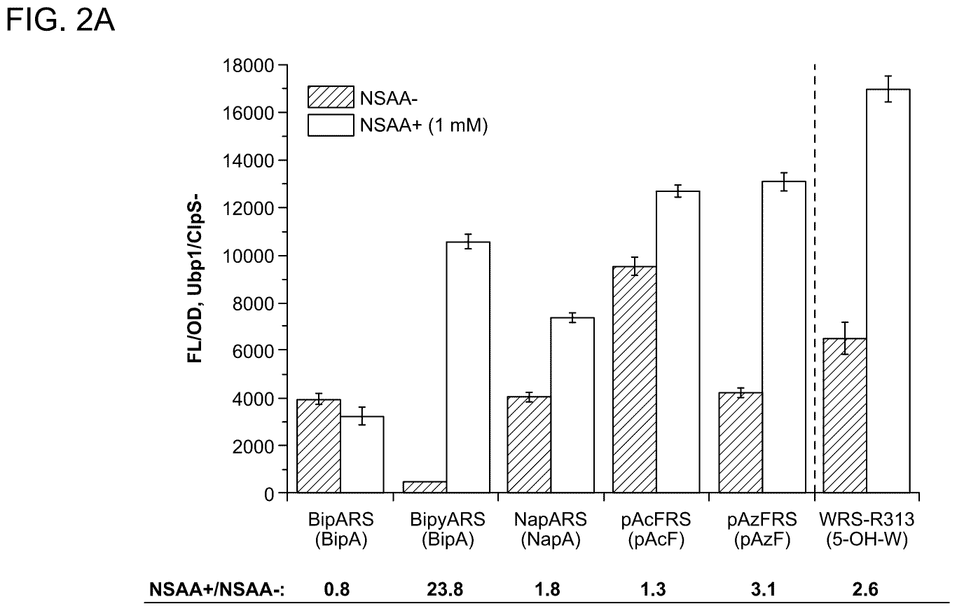

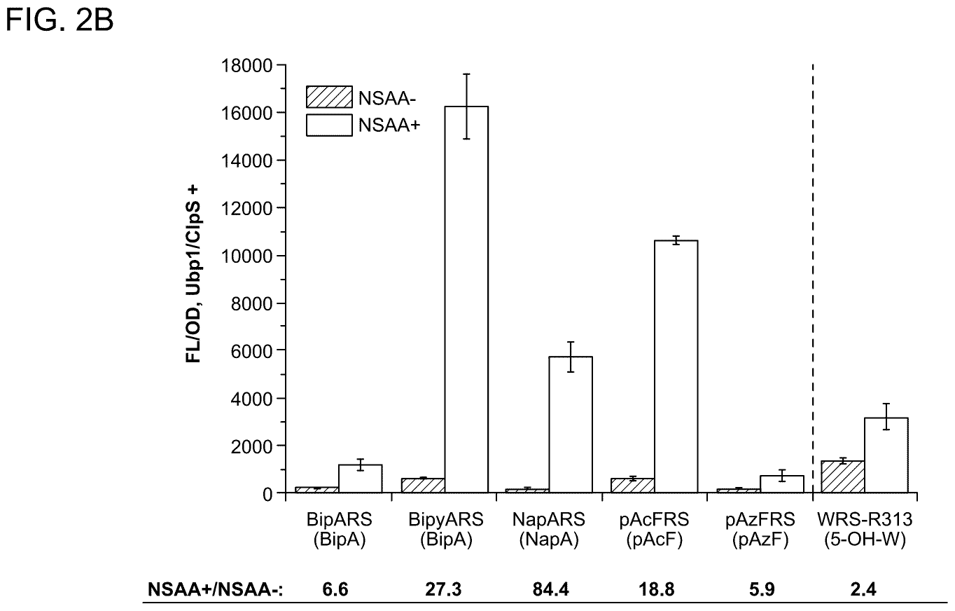

Finally, the native ClpS and the ClpP protease were overexpressed to determine their effect on the kinetics of GFP reporter degradation. The abundance of the GFP was measured as green fluorescence normalized by optical density (FL/OD) using a standard plate reader for fluorimetric and spectrophotometric assays. As shown in FIGS. 2A-2B, background protein synthesis (FL/OD) was nearly eliminated for all tested orthogonal translation systems upon expression of Ubp1/ClpS. Wild-type ClpS was not strictly selective as it recognized several small sized NSAAs and led to their degradation.

Since ClpS is endogenous to E. coli, it need not be overexpressed. However, overexpression, such as by use of an inducible promoter, increases the concentration of ClpS and therefore the ability of ClpS to discriminate between desired nonstandard amino acids and undesired standard amino acids or undesired NSAAs.

### EXAMPLE III

### Engineered ClpS V65I Mutant

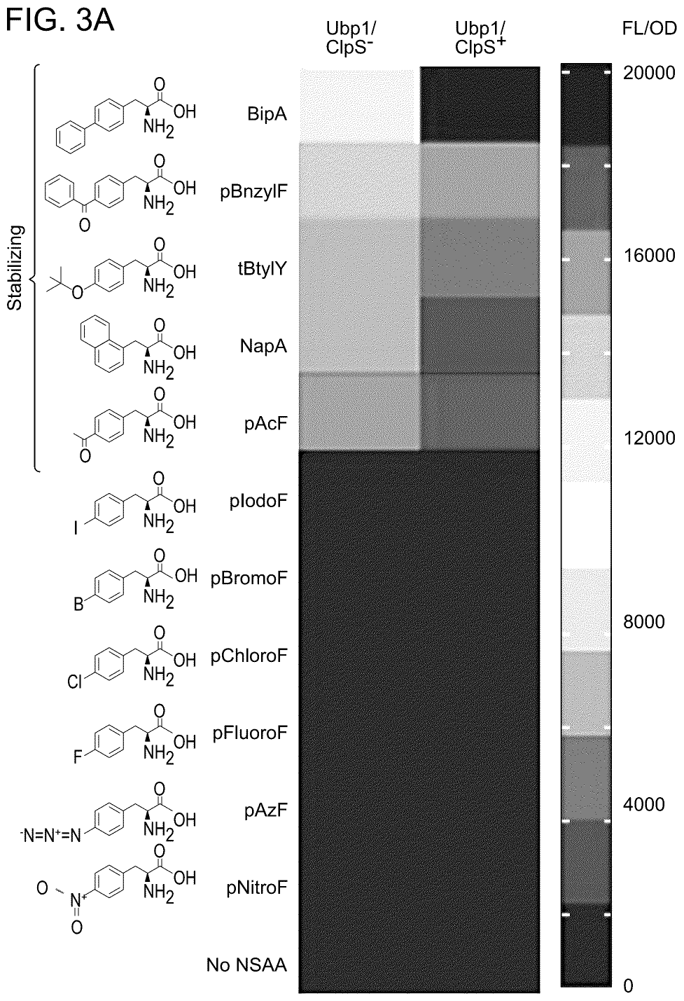

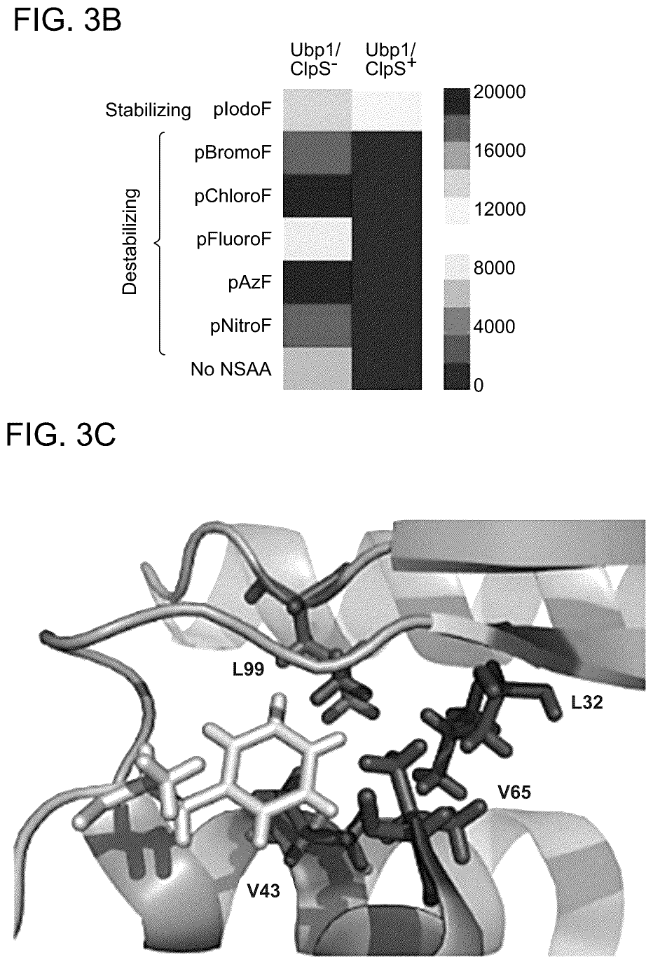

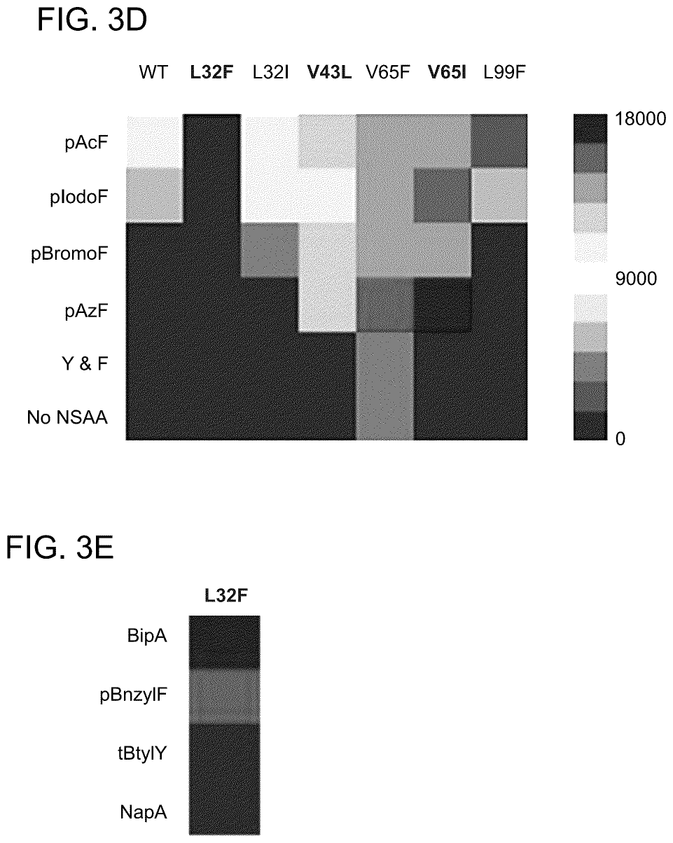

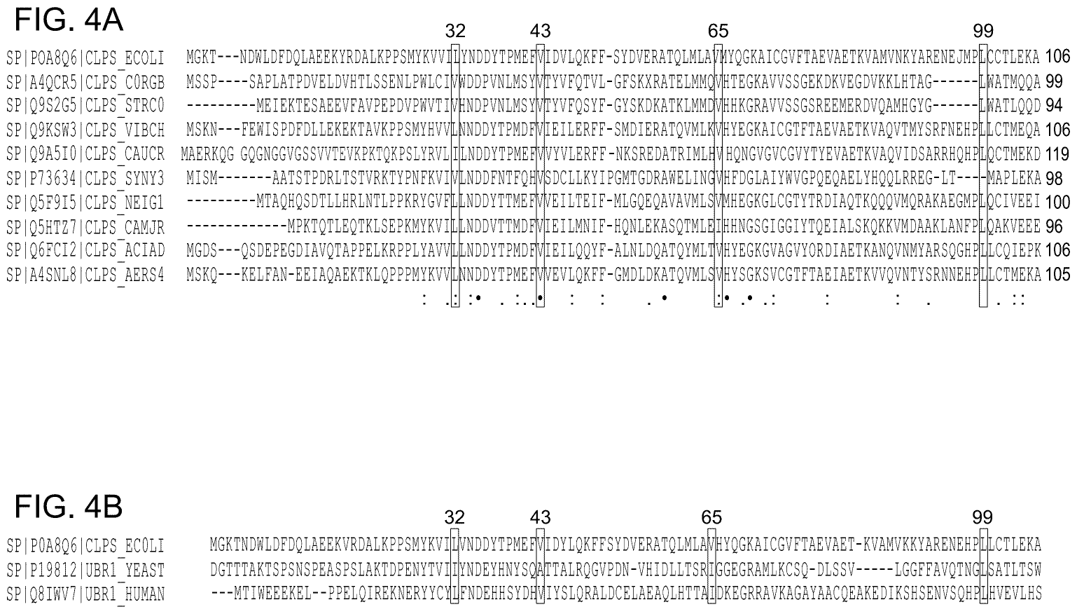

As shown in FIGS. 4A-4B, the crystal structure of the ClpS protein was used to rationally engineer ClpS variants that have tunable specificities for degrading different sizes of NSAAs, including undetectable degradation of tested analogs of Y/F while maintaining full degradation of Y/F with the ClpS_V65I mutant. The term “V65I” is a standard notation for describing a mutation where the amino acid I has been substituted for amino acid V at position 65. The ClpS_V65I mutant was capable of distinguishing between standard amino acids and their nonstandard amino acid counterparts when positioned at the N-terminus of a protein for purposes of degradation. One aspect of the present disclosure is to inactivate endogenous ClpS if the ClpS_V65I mutant is being used. Inactivation of ClpS prevents indiscriminate or nonselective degradation of proteins including smaller nonstandard amino acids. Another useful mutant is ClpS_L32 F, which causes biphenylalanine (bipA) and p-benzoylphenylalanine (pBnzylF) to be N-end stabilizing and all other tested NSAAs to be N-end destabilizing. In other words, the ClpS_L32F mutant can be used to discriminate between incorporated bipA and all other incorporated NSAAs other than pBnzylF (see FIG. 3E).

### EXAMPLE IV

### Local Sense Codon Reassignment

Aspects of the present disclosure are directed to local sense codon reassignment. Instead of using a stop codon for nonstandard amino acid incorporation, a sense codon can be used which creates competition for an N-terminal sense codon between a standard amino acid and a nonstandard amino acid. If the standard amino acid corresponding to the sense codon is destabilizing by the N-end rule, all of the proteins which do not include a sense codon reassigned to a nonstandard amino acid will be degraded. As shown in FIG. 1E with respect to the UAC codon which normally encodes tyrosine, when expression of Ubp1/ClpS is induced, nearly all of the GFP containing tyrosine gets degraded. If competition for the UAC codon with nonstandard amino acid is present, then nonstandard amino acid incorporation into the N-terminus of the GFP reporter protein can be detected by measuring the FL/OD increase.

### EXAMPLE V

### Materials and Methods

### Strains and Strain Engineering

E. coli strain C321.AA (CP006698.1), which was previously engineered to be devoid of UAG codons and RF1, was used as the E coli strain. E. coli strain C321.ΔA (CP006698.1) is described in M. J. Lajoie et al., Genomically Recoded Organisms Expand Biological Functions. Science (80-.). 342, 357-360 (2013) hereby incorporated by reference in its entirety. A starting reporter construct of an Ubiquitin-*-LFVQEL-sfGFP-His6x fusion_(“LFVQEL” and “His6x” disclosed as SEQ ID NOS 3 and 161, respectively) under an anhydrotetracycline (ATC) inducible expression system was genomically integrated using X Red recombineering as described in K. A. Datsenko, B. L. Wanner, One-step inactivation of chromosomal genes in Escherichia coli K-12 using PCR products. Proc Natl Acad Sci USA. 97, 6640-6645 (2000) and D. Yu et al., An efficient recombination system for chromosome engineering in Escherichia coli. Proc. Natd. Acad. Sci. U.S.A. 97, 5978-83 (2000) each of which are hereby incorporated by reference in its entirety and tolC negative selection using Colicin E1 as described in J. A. DeVito, Recombineering with tolC as a Selectable/Counter-selectable Marker: remodeling the rRNA Operons of Escherichia coli. Nucleic Acids Res. 36, e4 (2008) and C. J. Gregg et al., Rational optimization of tolC as a powerful dual selectable marker for genome engineering. Nucleic Acids Res. 42, 4779-4790 (2014) each of which are hereby incorporated by reference in its entirety. This resulted in strain C321.ΔA.Ubiq-UAG-sfGFP. Table 1 includes sequences of key constructs such as the reporter construct. Multiplex automatable genome engineering (MAGE) as described H. H. Wang et al., Programming cells by multiplex genome engineering and accelerated evolution. Nature. 460, 894-898 (2009) hereby incorporated by reference in its entirety was used to inactivate the endogenous mutS and clpS genes when needed and to add or remove UAG codons in the integrated reporter. In brief, overnight cultures were diluted 100-fold into 3 mL LB^{L} containing appropriate antibiotics and grown until mid-log. Lambda Red was induced in a shaking water bath (42° C., 300 rpm, 15 minutes), then induced culture tubes were cooled rapidly in an ice slurry for at least two minutes. Electrocompetent cells were prepared at 4° C. by pelleting 1 mL of culture (centrifuge at 16,000 rcf for 20 seconds) and washing the cell pellet twice with 1 mL ice cold deionized water (dH2O). Electrocompetent pellets were resuspended in 50 μL of dH2O containing the desired DNA. For MAGE oligonucleotides, 5 M of each oligonucleotide was used. Table 2 includes a list of all oligonucleotides used. For integration of dsDNA cassettes, 50 ng was used. Allele-specific colony PCR (ASC-PCR) was used to identify desired colonies resulting from MAGE as described in F. J. Isaacs et al., Precise manipulation of chromosomes in vivo enables genome-wide codon replacement. Science (80-.). 333, 348-353 (2011) hereby incorporated by reference in its entirety. Colony PCR was performed using Kapa 2G Fast HotStart ReadyMix according to manufacturer protocols and Sanger sequencing was performed by Genewiz to verify strain engineering. The strains C321.ΔA.Ubiq-UAG-sfGFP and C321.ΔA.Ubiq-UAG-sfGFP_UAG151 are available from Addgene.

TABLE 1
Sequences of key constructs

| Construct Name | Sequence | SEQ ID NO: |
|---|---|---|
| Ubiquitin-*- | ATGCAGATTTTTGTGAAGACTTTAACAGGTA | 4 |
| LFVQEL- | AGACGATTACCCTGGAGGTGGAGTCCTCGGA | |
| sfGFP_(″LFVQEL″ | CACCATCGATAATGTAAAATCAAAAATCCAA | |
| disclosed as SEQ ID | GATAAGGAAGGAATCCCTCCAGACCAGCAAC | |
| NO: 3) | GTCTGATTTTCGCAGGTAAACAACTGGAGGA | |
| | TGGTCGCACGCTTTCGGACTACAACATCCAG | |
| | AAAGAATCTACCCTTCATTTGGTTCTGCGTCT | |
| | GCGTGGAGGATAGTTGTTTGTGCAGGAGCTT | |
| | gcatccaagggcgaggagctctttactggcgtagtaccaattctcgtagag | |
| | ctcgatggcgatgtaaatggccataagttttccgtacgcggcgagggcga | |
| | gggcgatgcaactaacggcaagctcactctcaagtttatttgtactactggc | |
| | aagctcccagtaccatggccaactctcgtaactactctgacctatggcgtac | |
| | aatgtttttcccgctatccagatcacatgaagcaacatgatttttttaagtccg | |
| | caatgccagagggctatgtacaagagcgcactattagctttaaggatgatg | |
| | gcacctataagactcgcgcagaggtaaagtttgagggcgatactctcgtaa | |
| | atcgcattgagctcaagggcattgattttaaggaggatggcaatattctcgg | |
| | ccataagctggagtataatttcaattcccataatgtatacattaccgcagata | |
| | agcaaaagaatggcattaaggcgaattttaagattcgccataatgtggagg | |
| | atggctccgtacaactcgcagatcattatcaacaaaatactccaattggcga | |
| | tggcccagtactcctcccagataatcattatctctccactcaatccgtgctct | |
| | ccaaagatccaaatgagaagcgcgatcacatggtactcctggagtttgtaa | |
| | ctgcagcaggcattactcatggcatggatgagctctataagctcgagcacc | |
| | accaccaccaccactaa | |
| ClpS2_At gBlock | ATGTCTGATAGTCCTGTTGACTTAAAACCCA | 5 |
| | AGCCTAAAGTCAAGCCCAAATTAGAACGCCC | |
| | AAAACTTTACAAAGTCATGTTATTGAATGAT | |
| | GATTATACACCACGCGAATTTGTGACGGTAG | |
| | TCCTTAAAGCGGTGTTTCGTATGTCAGAGGA | |
| | CACTGGTCGCCGTGTAATGATGACAGCACAT | |
| | CGTTTTGGTTCGGCGGTGGTGGTCGTTTGTGA | |
| | ACGTGACATTGCAGAGACGAAAGCCAAGGA | |
| | GGCGACCGACTTGGGGAAGGAAGCAGGTTTT | |
| | CCTTTGATGTTCACGACTGAGCCCGAGGAGT | |
| | AA | |
| pAzFRS.1.t1 | GTTATGcactacGATggtgttgacgttTACgttggtggtatggaa | 6 |
| gBlock | cagcgtaaaatccacatgctggcgcgtgaactgctgccgaaaaaagttgtt | |
| | tgcatccacaacccggttctgaccggtctggacggtgaaggtaaaatgtct | |
| | tcttctaaaggtaacttcatcgcggttgacgactctccggaagaaatccgtg | |
| | cgaaaatcaaaaaagcgtactgcccggcgggtgttgttgaaggtaacccg | |
| | atcatggaaatcgcgaaatacttcctggaatacccgctgaccatcaaaGG | |
| | T | |
| Ubp1_Sc_trunc | ATGGGGAGTGGGTCTTTCATTGCTGGGCTTGT | 7 |
| | CAACGATGGTAATACGTGTTTTATGAACTCG | |
| | GTTCTTCAGTCCCTTGCTAGTAGCCGTGAACT | |
| | TATGGAGTTTTTGGATAATAATGTAATCCGTA | |
| | CATATGAAGAAATTGAACAGAACGAGCACA | |
| | ATGAGGAAGGTAATGGCCAAGAGAGCGCAC | |
| | AAGATGAGGCAACTCACAAAAAAAACACTC | |
| | GCAAGGGAGGTAAGGTCTATGGGAAGCATA | |
| | AAAAGAAATTAAACCGCAAATCTTCTAGCAA | |
| | GGAAGACGAAGAAAAGTCGCAAGAACCAGA | |
| | CATTACGTTTTCGGTGGCGTTGCGTGATCTGC | |
| | TGAGCGCATTAAATGCTAAGTATTATCGCGA | |
| | CAAACCCTACTTTAAGACTAACTCTTTATTAA | |
| | AAGCGATGAGCAAGTCCCCGCGCAAAAATAT | |
| | CTTGCTTGGGTACGATCAAGAAGACGCTCAG | |
| | GAATTTTTTCAAAACATTCTTGCGGAGTTAGA | |
| | ATCTAATGTCAAGTCGTTAAACACAGAAAAG | |
| | CTTGATACTACACCGGTAGCCAAGTCCGAAC | |
| | TTCCAGACGATGCTCTGGTTGGCCAATTAAA | |
| | CCTTGGTGAGGTAGGCACCGTGTACATTCCC | |
| | ACAGAACAAATTGACCCCAATTCGATTTTAC | |
| | ATGACAAATCGATTCAAAACTTTACCCCCTTT | |
| | AAACTGATGACCCCGTTGGATGGGATCACGG | |
| | CTGAGCGCATCGGCTGCCTGCAATGCGGAGA | |
| | GAACGGGGGAATTCGCTACAGTGTTTTCAGC | |
| | GGATTAAGTTTGAACCTGCCGAATGAAAATA | |
| | TTGGAAGCACTCTTAAACTGTCCCAGTTACTG | |
| | TCCGATTGGTCGAAACCCGAGATTATCGAGG | |
| | GTGTTGAATGCAACCGTTGCGCTTTAACAGC | |
| | TGCGCACTCACACTTGTTTGGCCAATTAAAG | |
| | GAGTTTGAGAAGAAACCTGAAGGCTCGATTC | |
| | CCGAAAAACTTATTAATGCCGTAAAGGACCG | |
| | CGTGCACCAGATCGAAGAGGTCTTGGCAAAG | |
| | CCGGTTATCGACGATGAAGATTATAAAAAAT | |
| | TGCATACTGCGAATATGGTCCGCAAGTGTTC | |
| | AAAAAGTAAACAAATTCTTATCTCTCGTCCA | |
| | CCACCTTTGTTGTCTATTCATATCAACCGCTC | |
| | TGTTTTCGACCCGCGCACCTACATGATTCGCA | |
| | AGAACAACTCCAAGGTTTTGTTCAAGTCACG | |
| | CTTGAACCTGGCACCCTGGTGCTGTGATATC | |
| | AACGAAATCAATCTTGACGCACGCCTTCCGA | |
| | TGTCGAAGAAGGAAAAAGCAGCTCAACAAG | |
| | ATTCTTCTGAAGACGAGAACATTGGCGGAGA | |
| | GTACTATACTAAATTGCATGAACGTTTTGAG | |
| | CAGGAGTTTGAAGATTCTGAAGAAGAGAAG | |
| | GAATACGATGATGCAGAGGGTAATTATGCAT | |
| | CGCATTATAACCATACCAAGGACATCTCCAA | |
| | CTACGATCCATTGAATGGAGAAGTCGACGGT | |
| | GTGACTTCCGATGATGAGGATGAATACATTG | |
| | AAGAGACAGACGCGTTGGGGAATACCATCA | |
| | AAAAACGTATTATTGAACACTCCGACGTGGA | |
| | GAACGAAAACGTGAAGGATAATGAAGAACT | |
| | TCAGGAGATCGATAACGTTAGCTTGGATGAG | |
| | CCAAAAATTAATGTCGAGGACCAGCTTGAAA | |
| | CGAGTTCTGATGAGGAAGACGTTATTCCTGC | |
| | TCCACCCATCAACTACGCTCGCAGCTTTAGTA | |
| | CGGTCCCAGCGACCCCTTTAACTTACTCTTTG | |
| | CGCAGCGTCATCGTGCACTATGGGACTCACA | |
| | ACTACGGACATTATATTGCATTTCGCAAGTAT | |
| | CGTGGATGTTGGTGGCGCATCTCCGATGAGA | |
| | CGGTCTATGTGGTAGATGAGGCCGAAGTACT | |
| | GTCAACACCGGGGGTATTTATGCTTTTCTACG | |
| | AGTATGATTTCGACGAGGAGACCGGAAAAAT | |
| | GAAAGACGACTTAGAAGCTATCCAGAGCAAT | |
| | AATGAGGAAGATGACGAGAAAGAACAGGAA | |
| | CAGAAGGGTGTCCAGGAGCCAAAAGAATCC | |
| | CAGGAGCAAGGCGAAGGCGAAGAACAAGAA | |
| | GAAGGGCAAGAGCAAATGAAATTTGAGCGT | |
| | ACGGAGGATCATCGCGACATTTCAGGGAAGG | |
| | ATGTGAATTAA | |

TABLE 2
Oligonucleotides used

| Oligo Name | Sequence | SEQ ID NO: |
|---|---|---|
| pZE21-seq-F | CCATTATTATCATGACATTAACC | 8 |
| pZE21-seq-R | GGATTTGTCCTACTCAGGAG | 9 |
| AARS-seq-F | CTTTTTATCGCAACTCTC | 10 |
| Ubiquitin + N-degron-F | TTAAAGAGGAGAAATTAACTATGCAGAT | 11 |
| | TTTTGTGAAGACT | |
| Ubiquitin + N-degron-R | AGCTCCTCGCCCTTGGATGCAAGCTCCTG | 12 |
| | CACAAACAAGT | |
| pEVOLbbone_Ubp1-F | CAGGGAAGGATGTGAATTAATAAGTCGA | 13 |
| | CCATCATCATCA | |
| pEVOLbbone_Ubp1-R | ATGAAAGACCCACTCCCCATAGATCTAA | 14 |
| | TTCCTCCTGTTAGC | |
| Ubp1-P1-F | TAACAGGAGGAATTAGATCTATGGGGAG | 15 |
| | TGGGTCTTTCAT | |
| Ubp1-P1-R | TCAAGCGTGACTTGAACAAAACCTTGGA | 16 |
| | GTTGTTCTTGCG | |
| Upb1-P2-F | CGCAAGAACAACTCCAAGGTTTTGTTCA | 17 |
| | AGTCACGCTTGA | |
| Upb1-P2-R | TGATGATGATGGTCGACTTATTAATTCAC | 18 |
| | ATCCTTCCCTGA | |
| pUbi-*-Ndeg-GFP-F | TGCGTCTGCGTGGAGGATAGTTGTTTGTG | 19 |
| | CAGGAGCTTGC | |
| pUbi-*-Ndeg-GFP-R | AAGCTCCTGCACAAACAACTATCCTCCAC | 20 |
| | GCAGACGC | |
| Ubp1_int-seq-F | GCTTGGGTACGATCAAGAAG | 21 |
| Ubp1_int-seq-R | CCTTGGTATGGTTATAATGCG | 22 |
| pZE21bbone4Ubp1-F | CAGGGAAGGATGTGAATTAAAAGCTTGA | 23 |
| | TGGGGGATCCCA | |
| pZE21bbone4Ubp1-R | ATGAAAGACCCACTCCCCATGGTACCTTT | 24 |
| | CTCCTCTTTAATGAAT | |
| Ubp1-ins-F | TTAAAGAGGAGAAAGGTACCATGGGGAG | 25 |
| | TGGGTCTTTCAT | |
| Ubp1-ins-R | TGGGATCCCCCATCAAGCTTTTAATTCAC | 26 |
| | ATCCTTCCCTGA | |
| UbiGFPins-F | TAAAGAGGAGAAAGGTACCATGCAGATT | 27 |
| | TTTGTGAAGACTTTAAC | |
| UbiGFPins-R | TGGGATCCCCCATCAAGCTTTTAGTGGTG | 28 |
| | GTGGTGGTGGT | |
| pZEbbone4UbiGFP-F | ACCACCACCACCACCACTAAAAGCTTGA | 29 |
| | TGGGGGATCCCA | |
| pZEbbone4UbiGFP-R | GTCTTCACAAAAATCTGCATGGTACCTTT | 30 |
| | CTCCTCTTTAATGAAT | |
| reporter_to_genome-F | TTACGGGCTAATTACAGGCAGAAATGCG | 31 |
| | TGATGTGTGCCACACTTGTTGATCCCTAT | |
| | CAGTGATAGAGATTGAC | |
| reporter_to_genome-R | CCAGCGGGCTAACTTTCCTCGCCGGAAG | 32 |
| | AGTGGTTAACAAAATAGTAACGTCACCG | |
| | ACAAACAACAGATAAAAC | |
| SIR-seq-F | CCAAAGTGAGTTGAGTATAAC | 33 |
| SIR-seq-R | TTTCTCCTTATTATCAATGC | 34 |
| r2g-extend-F | GCCGCAGCAAGCCAAAGTGAGTTGAGTA | 35 |
| | TAACGCAAATTTGCTACTGGTCCGATGGG | |
| | TGCAATGGTCTGAATTACGGGCTAATTAC | |
| | AGGC | |
| r2g-extend-R | AACGCAATCGCAACCGCTAAACCACTGG | 36 |
| | CCATGTGCACGAGTTTCATTCATTTCTCC | |
| | TTATTATCAATGCACCAGCGGGCTAACTT | |
| | TC | |
| MAGE_*toS | t*a*aagagctcctcgcccttggatgcAAGCTCCTGCAC | 37 |
| | AAACAACgATCCTCCACGCAGACGCAGA | |
| | ACCAAATGAAGGGTAGATTCTTTCT | |
| asPCR-S-F | CGTCTGCGTGGAGGATC | 38 |
| asPCR-*-F | CGTCTGCGTGGAGGATA | 39 |
| pZE-Ubp1bbone4ClpP- | TTCTGACCCATCGTAATTAAaagcttgatggggg | 40 |
| F | atccca | |
| pZE-Ubp1bbone4ClpP- | tGGTATATCTCCTTTTATTATTAATTCACA | 41 |
| R | TCCTTCCCTGAAAT | |
| clpPins-F | GTGAATTAATAATAAAAGGAGATATACC | 42 |
| | atgTCATACAGCGGCGA | |
| clpPins-R | tgggatcccccatcaagcttTTAATTACGATGGGTC | 43 |
| | AGAATCG | |
| pEVOLtRNA-p1-F | ctgccaacttactgatttagtgtatgatggtgtttttgagg | 44 |
| pEVOLRNA-p1-R | gccgcttagttagccgtgcaaacttatatcgtatggggctg | 45 |
| pEVOLtRNA-p2-F | agccccatacgatataagtttgcacggctaactaagcggc | 46 |
| pEVOLtRNA-p2-R | ctcaaaaacaccatcatacactaaatcagtaagttggcagcatca | 47 |
| pZE-Ubp1bbone4ClpS- | TGTGTACGCTAGAAAAAGCCTAAaagcttgat | 48 |
| F | gggggatc | |
| pZE-Ubp1bbone4ClpS- | GTTCGTTTTACCcatGGTATATCTCCTTTTA | 49 |
| R | TTATTAATTCACAT | |
| ClpSins-F | ATAATAAAAGGAGATATACCatgGGTAAA | 50 |
| | ACGAACGACTG | |
| ClpSins-R | gatcccccatcaagcttTTAGGCTTTTTCTAGCGTA | 51 |
| | CACA | |
| AARSlibraryins-F | tactgtttctccatacccgtttttttgggctaacaggaggaattagatct | 52 |
| pEVOLbbone4lib-R | agatctaattcctcctgttagcc | 53 |
| mutS_null_mut-2* | A*C*CCCATGAGTGCAATAGAAAATTTCG | 54 |
| | ACGCCCATACGCCCATGATGCAGCAGTG | |
| | ATAGTCGCTGAAAGCCCAGCATCCCGAG | |
| | ATCCTGC | |
| mutS_null_revert-2* | A*C*CCCATGAGTGCAATAGAAAATTTCG | 55 |
| | ACGCCCATACGCCCATGATGCAGCAGTA | |
| | TCTCAGGCTGAAAGCCCAGCATCCCGAG | |
| | ATCCTGC | |
| mutS-2_ascPCR_wt-F | CCATGATGCAGCAGTATCTCAG | 56 |
| mutS-2_ascPCR_mut-F | CCATGATGCAGCAGTGATAGTC | 57 |
| mutS-2_ascPCR-R | AGGTTGTCCTGACGCTCCTG | 58 |
| ASPCR-151UAG-F | GTATAATTTCAATTCCCATAATGTATAG | 59 |
| ASPCR-151UAC-F | GTATAATTTCAATTCCCATAATGTATAC | 60 |
| ASPCR-151-R | ctcgagcttatagagctcatc | 61 |
| Remove151UAG- | c*t*taaaattcgccttaatgccattcttttgcttatctgcggtaatgtat | 62 |
| MAGE_corrected | acattatgggaattgaaattatactccagcttatggccgag | |
| ClpS.inact-MAGE | C*T*TTTTCTTCCGCCAGTTGATCAAAGTC | 63 |
| | CAGCCAGTCGTTCtaTTatCaCATTGTCAGT | |
| | TATCATCTTCGGTTACGGTTATCGGCAGA | |
| | AC | |
| ASPCR-ClpS_WT-F | CCGATAACCGTAACCGAAGATGATAACT | 64 |
| | GACAATGG | |
| ASPCR-ClpS.inact-F | CCGATAACCGTAACCGAAGATGATAACT | 65 |
| | GACAATGT | |
| ASPCR-ClpS-R | CGTACTTGTTCACCATCGCCACTTTGGT | 66 |
| pZE-U- | CGACTGAGCCCGAGGAGTAAaagcttgatgggg | 67 |
| bbone4ClpS2_At-F | gatccca | |
| pZE-U- | TCAACAGGACTATCAGACATGGTATATCT | 68 |
| bbone4ClpS2_At-R | CCTTTTATTATTAATTCACATCC | |
| ClpS2_At-ins-F | ATAATAAAAGGAGATATACCATGTCTGA | 69 |
| | TAGTCCTGTTGACTT | |
| ClpS2_At-ins-R | tgggatcccccatcaagcttTTACTCCTCGGGCTCAG | 70 |
| | TCG | |
| ClpS_M40A-F | ATGATGATTACACTCCGGCGGAGTTTGTT | 71 |
| | ATTGACGTGT | |
| ClpS_M40A-R | CGTCAATAACAAACTCCGCCGGAGTGTA | 72 |
| | ATCATCATTGAC | |
| pOSIPbbone-F | taacctaaactgacaggcat | 73 |
| pOSIPbbone-R | ttccgatccccaattcct | 74 |
| pEVOL-araC-seq-1 | GGATCATTTTGCGCTTCAG | 75 |
| pEVOL-araC-seq-2 | GAATATAACCTTTCATTCCC | 76 |
| PylRSmiddle-seq | GTGTTTCGACTAGCATTTC | 77 |
| PylRSend-seq | GGTCAAACATGATTTCAAAAAC | 78 |
| pEVOLCmR-seq-R | caacagtactgcgatgag | 79 |
| upstreamClpS-F | GCAAATAAGCTCTTGTCAGC | 80 |
| ClpS_L32-NTC-F | CATCTATGTATAAAGTGATANTCGTCAAT | 81 |
| | GATGATTACACTCCG | |
| ClpS_32-R | TATCACTTTATACATAGATG | 82 |
| ClpS-V43-NTT-F | ATTACACTCCGATGGAGTTTNTTATTGAC | 83 |
| | GTGTTACAAAAATTC | |
| ClpS_43-R | AAACTCCATCGGAGTGTAAT | 84 |
| ClpS_V65-NTT-F | CAACGCAATTGATGCTCGCTNTTCACTAC | 85 |
| | CAGGGGAAGG | |
| ClpS_65-R | AGCGAGCATCAATTGCGTTG | 86 |
| ClpS_L99-NTC-F | CGAGGGAGAATGAGCATCCANTCCTGTG | 87 |
| | TACGCTAGAAAAAGC | |
| ClpS_99-R | TGGATGCTCATTCTCCCTCG | 88 |
| Alt_ClpS-R_forL99 | gcggatttgtcctactcag | 89 |
| AARS-inducible-only-F | gctaacaggaggaattagatct | 90 |
| AARS-inducible-only-R | ttgataatctaacaaggattatggg | 91 |
| pEVOLbbone-Ind-only- | cccataatccttgttagattatcaaaggcattttgctattaaggg | 92 |
| F | | |
| pEVOL-bbone-ind- | agatctaattcctcctgttagc | 93 |
| only-R | | |
| protosens-bbone-F | TAACTCGAGGCTGTTTTGG | 94 |
| protosens-bbone-R | CATATGTATATCTCCTTGTGCATC | 95 |
| Ubp1ClpS4protosens-F | GATGCACAAGGAGATATACATATGGGGA | 96 |
| | GTGGGTCTTTCAT | |
| Ubp1ClpS4protosens-R | CCAAAACAGCCTCGAGTTAGGCTTTTTCT | 97 |
| | AGCGTACA | |
| pAzFRS.1.t1-ins-F | acccgatcatgcaggttaacGTTATGcactacGATggtgt | 98 |
| pAzFRS.1.t1-ins-R | tcaccaccgaatttttccggACCtttgatggtcagcg | 99 |
| bbone4pAzFRS.1.t1-F | ccggaaaaattcggtggtga | 100 |
| bbone4pAzFRS.1.t1-R | gttaacctgcatgatcgggt | 101 |
| pZEbbone4tetR-F | acgctctcctgagtaggac | 102 |
| pZEbbone4tetR-R | tcaccgacaaacaacagataaaac | 103 |
| TetR-ins-F | tatctgttgtttgtcggtgaacgtctcattttcgccagat | 104 |
| TetR-ins-R | gtcctactcaggagagcgtagtgtcaactttatggctagc | 105 |
| pDULE-ABK-bbone-F | cgacctgaatggaagcc | 106 |
| pDULE-ABK-bbone-R | catacacggtgcctgac | 107 |
| CmRins4pDULE-F | aacgcagtcaggcaccgtgtatggagaaaaaaatcactggatatac | 108 |
| CmR4pDULE-R | gccggcttccattcaggtcgaaaaaattacgccccgc | 109 |
| pCNFRS-65-67-70- | CAAAATGCTGGATTTGATATAATTATANN | 110 |
| NNK-F | KTTGNNKGATTTANNKGCCTATTTAAACC | |
| | AGAAAGGAGAG | |
| pCNFRS-65-R | TATAATTATATCAAATCCAGCATTTTGTA | 111 |
| | AATC | |
| pCNFRS-108-109-114- | GGCAAAATATGTTTATGGAAGTGAANNK | 112 |
| NNK-F | NNKCTTGATAAGGATNNKACACTGAATG | |
| | TCTATAGATTGGC | |
| pCNFRS-108-R | TTCACTTCCATAAACATATTTTGCC | 113 |
| pCNFRS-155-157-161- | GAAGTTATCTATCCAATAATGNNKGTTN | 114 |
| NNK-F | NKGGTGCTCATNNKCTTGGCGTTGATGTT | |
| | GCAG | |
| pCNFRS-155-R | CATTATTGGATAGATAACTTCAGCAAC | 115 |
| libraryINS-seq-R | CGCATCAGGCAATTTAGC | 116 |
| BipARS_P144Q-F | cgcgcgtgaagacgaaaaccagaaagttgcggaagttatctac | 117 |
| BipARS_P144Q-R | ggttttcgtcttcacgcg | 118 |
| BipARS_N157K-F | tacccgatcatgcaggttaaaggtatccactacaaaggtgttg | 119 |
| BipARS_N157K-R | ttaacctgcatgatcgggta | 120 |
| BipARS_R181C-F | gtaaaatccacatgctggcgtgtgaactgctgccgaaa | 121 |
| BipARS_R181C-R | cgccagcatgtggatttta | 122 |
| BipARS_I255F-F | tcctggaatacccgctgaccttcaaacgtccggaaaaattc | 123 |
| BipARS_I255F-R | ggtcagcgggtattccag | 124 |
| BipARS_E259V-F | gctgaccatcaaacgtccggtaaaattcggtggtgacctg | 125 |
| BipARS_E259V-R | ccggacgtttgatggtc | 126 |
| BipARS_P284S-F | tcaaaaacaaagaactgcactcgatgcgtctgaaaaacg | 127 |
| BipARS_P284S-R | gtgcagttctttgtttttgaac | 128 |
| pEVOLbbone4libv2-F | ctgcagtttcaaacgctaaattg | 129 |
| AARSlibraryinsv2-R | taggcctgataagcgtagcgcatcaggcaatttagcgtttgaaactg | 130 |
| | cag | |
| BipARS_G257R-F | aatacccgctgaccatcaaacgtccggaaaaattcggtg | 131 |
| BipARS_G257R-R | accaccgaatttttccggacgtttgatggtcagcgggtat | 132 |
| BipARS-100AA-F | gcgaaatacgtttacggttc | 133 |
| BipARS-100AA-R | gaaccgtaaacgtatttcgc | 134 |
| BipARS-200AA-F | ggacggtgaaggtaaaatgtc | 135 |
| BipARS-200AA-R | gacattttaccttcaccgtcc | 136 |
| pZErepbbone4pylT-F | cggcgccagggttgtttttcacgctctcctgagtaggaca | 137 |
| pZErepbbone4pylT-R | ttccattcaggtcgaaaaaaagtgtcaactttatggctagc | 138 |
| pylTpDULE-F | ttttttcgacctgaatggaagc | 139 |
| pylTpDULE-R | gaaaaacaaccctggcgc | 140 |
| pZEbbone4pylTonly-F | cggcgccagggttgtttttcacgctctcctgagtaggaca | 141 |
| pZEbbone4pylTonly-R | ttccattcaggtcgaaaaaactcgaggtgaagacgaaagg | 142 |
| ClpS-Lib-F | ACATTTCAGGGAAGGATGTGAATTAATA | 143 |
| | ATAAAAGGAGATATACC | |
| ClpS-Lib-R | gcgtaccatgggatcccccatcaagcttTTA | 144 |
| pZEbbone4ClpSlib-F | TAAaagcttgatgggggatc | 145 |
| pZEbbone4ClpSlib-R | GGTATATCTCCTTTTATTATTAATTCACAT | 146 |
| | CC | |
| ClpS-Lib-Seq | GGATCATCGCGACATTTC | 147 |

### Plasmids and Plasmid Construction

Two copies of orthogonal MjTyrRS-derived AARSs and

$${tRNA}_{CUA}^{Tyr}$$

were provided in pEVOL plasmids by Dr. Peter Schultz (Scripps Institute) and as described in T. S. Young, I. Ahmad, J. A. Yin, P. G. Schultz, An Enhanced System for Unnatural Amino Acid Mutagenesis in E. coli. J. Mol. Biol. 395, 361-374 (2010) hereby incorporated by reference in its entirety. The pEVOL plasmids were maintained using chloramphenicol. Original plasmids harboring two AARS copies were used for synthetase promiscuity comparison experiments as shown in FIGS. 1A-1E, FIGS. 2A-2B and FIGS. 3A-3E. For generation and characterization of synthetase variants, plasmids harboring only one AARS copy under inducible expression were constructed using Gibson assembly as described in D. G. Gibson et al., Enzymatic assembly of DNA molecules up to several hundred kilobases. Nat. Methods. 6, 343-345 (2009) hereby incorporated by reference in its entirety. Plasmids containing PylIFRS an

$${tRNA}_{CUA}^{Pyl}$$

were provided by Professor Dieter Soll (Yale University) as described in L.-T. Guo et al., Polyspecific pyrrolysyl-tRNA synthetases from directed evolution, doi:10.1073/pnas.1419737111 hereby incorporated by reference in its entirety. The PylIFRS was retained on its original pCDF plasmid but the tRNA was cloned into the pZE21 vector for higher expression. The pCDF plasmid was maintained using spectinomycin and the pZE21 plasmid was maintained using kanamycin. The Pyl-ABK and

$${tRNA}_{CUA}^{Pyl} AARS / tRNA$$

pair were provided by Dr. Peter Schultz (pDULE-ABK, Addgene plasmid #49086) as described in H. Ai, W. Shen, A. Sagi, P. R. Chen, P. G. Schultz, Probing Protein-Protein Interactions with a Genetically Encoded Photo-crosslinking Amino Acid. ChemBioChem. 12, 1854-1857 (2011) hereby incorporated by reference in its entirety. The antibiotic resistance marker on the pDULE plasmid was swapped for chloramphenicol using Gibson assembly. The ScWRS-R3-13 AARS was synthesized as codon-optimized for expression in E. coli and cloned into the pEVOL plasmid along with its associated tRNA as described in R. A. Hughes, A. D. Ellington, Rational design of an orthogonal tryptophanyl nonsense suppressor tRNA. Nucleic Acids Res. 38, 6813-6830 (2010) and J. W. Ellefson et al., Directed evolution of genetic parts and circuits by compartmentalized partnered replication. Nat. Biotechnol. 32, 97-101 (2014) each of which is hereby incorporated by reference in its entirety. In all cases, tRNA is constitutively expressed and AARS expression is either arabinose inducible or constitutive.

An N-terminally truncated form of the ubp1 gene from Saccharomyces cerevisiae as described in J. W. Tobias, A. Varshavsky, Cloning and functional analysis of the ubiquitin-specific protease gene UBP1 of Saccharomyces cerevisiae. J. Biol. Chem. 266, 12021-8 (1991) or A. Wojtowicz et al., Expression of yeast deubiquitination enzyme UBP1 analogues in E. coli. Microb. Cell Fact. 4, 1-12 (2005) each of which is hereby incorporated by reference in its entirety was synthesized as codon-optimized for expression in E. coli and cloned into the pZE21 vector (Kanamycin resistance, ColE1 origin, TET promoter) (Expressys). The E. coli genes clpS and clpP were amplified from E. coli MG1655 and cloned into artificial operons downstream of the ubp1 gene in the pZE21 vector using Gibson assembly. Artificial operons were created by inserting the following RBS sequence between the ubp1 and clp genes: TAATAAAAGGAGATATACC (SEQ ID NO: 148). This RBS was originally designed using the RBS calculator (see H. M. Salis, E. A. Mirsky, C. A. Voigt, Automated design of synthetic ribosome binding sites to control protein expression. Nat Biotech. 27, 946-950 (2009)) and previously validated in the context of another artificial operon as described in A. M. Kunjapur, Y. Tarasova, K. L. J. Prather, Synthesis and Accumulation of Aromatic Aldehydes in an Engineered Strain of Escherichia coli. J. Am. Chem. Soc. 136, 11644-54 (2014) hereby incorporated by reference in its entirety. Rational engineering of ClpS variants was performed by dividing the clpS gene into two amplicons where the second amplicon contained a degenerate NTC or NTT sequence in the oligo corresponding to each codon of interest. The four initial positions of interest in the clpS gene correspond to amino acids 32, 43, 65, and 99. In each case, Gibson assembly was used to ligate both amplicons and the backbone plasmid. The pZE/Ubp1/ClpS and pZE/Ubp1/ClpS_V65I plasmids are available from Addgene. The ubp1/clpS_V65I operon was also placed under weak constitutive expression and integrated using Clonetegration as described in F. St-Pierre et al., One-step cloning and chromosomal integration of DNA. ACS Synth. Biol. 2, 537-541 (2013). This strain (C321.ΔA.Nendint) was used as the host for FACS experiments.

### Culture Conditions

Cultures for general culturing used herein were grown in LB-Lennox medium (LB^{L}: 10 g/L bacto tryptone, 5 g/L sodium chloride, 5 g/L yeast extract). Cultures for experiments in FIGS. 3A-3E were grown in 2X YT medium (2XYT: 16 g/L bacto tryptone, 10 g/L bacto yeast extract, 5 g/L sodium chloride) given improved observed final culture densities compared to LB^{L} upon expression of ClpS variants. Unless otherwise indicated, all cultures were grown in biological triplicate in 96-well deep-well plates in 300 μL culture volumes at 34C and 400 rpm.

### NSAA Incorporation Assays

Strains harboring integrated GFP reporters and AARS/tRNA plasmids were inoculated from frozen stocks of biological triplicate and grown to confluence overnight in deep well plates. Experimental cultures were inoculated at 1:100 dilution in either LB^{L} or 2XYT media supplemented with chloramphenicol, arabinose, and the appropriate NSAA. Cultures were incubated at 34° C. to an OD₆₀₀ of 0.5-0.8 in a shaking plate incubator at 400 rpm (˜4-5 h). GFP expression was induced by addition of anhydrotetracycline, and cells were incubated at 34° C. for an additional 16-20 h.

All assays were performed in 96-well plate format. Cells were centrifuged at 5,000 g for 3 min, washed with PBS, and resuspended in PBS after a second spin. GFP fluorescence was measured on a Biotek spectrophotometric plate reader using excitation and emission wavelengths of 485 and 525 nm. Fluorescence signals were corrected for autofluorescence as a linear function of OD₆₀₀ using the parent C321.ΔA strain that does not contain a reporter. Fluorescence was then normalized by the OD₆₀₀ reading to obtain FL/OD.

### Chemicals

NSAAs used in this study were purchased from PepTech Corporation, Sigma Aldrich, Santa Cruz Biotechnology, and Toronto Research Chemicals. The following NSAAs were purchased: L-4,4-Biphenylalanine (BipA), L-4-Benzoylphenylalanine (pBenzoylF), O-tert-Butyl-L-tyrosine (tButylY), L-2-Naphthylalanine (NapA), L-4-Acetylphenylalanine (pAcF), L-4-Iodophenylalanine (pIF), L-4-Bromophenylalanine (pBromoF), L-4-Chlorophenylalanine (pChloroF), L-4-Fluorophenylalanine (pFluoroF), L-4-Azidophenylalanine (pAzF), L-4-Nitrophenylalanine, L-4-Cyanophenylalanine, L-3-Iodophenylalanine, L-phenylalanine, L-tyrosine, L-tryptophan, D-phenylalanine, D-tyrosine, and 5-Hydroxytryptophan.Solutions of NSAAs (50 or 100 mM) were made in 10-50 mM NaOH.

### Library Generation

Error-prone PCR (EP-PCR) is the method of choice for introducing random mutations into a defined segment of DNA that is too long to be chemically synthesized as a degenerate sequence. EP-PCR was performed using the GeneMorph II Random Mutagenesis Kit (Stratagene Catalog #200550), following manufacturer instructions to obtain approximately an average of 2-4 DNA mutations per library member. To generate libraries of MjTyrRS-derived AARSs, roughly 175 ng of PCR template was used in each 25 μL of PCR mix containing primers that have roughly 40 base pairs of homology flanking the AARS coding region. The reaction mixture was subject to 30 cycles with Tm of 63° C. and extension time of 1 min. Four separate 25 μL EP-PCR reactions were performed per AARS and then pooled. Plasmid backbone PCRs were performed using KOD Xtreme Hot Start Polymerase (Millipore Catalog #71795). Both PCR products were isolated by 1.5% agarose gel electrophoresis and Gibson assembled in 8 parallel 20 uL volumes per library. Assemblies were pooled, washed by ethanol precipitation, and resuspended in 50 L of dH₂0, which was drop dialyzed (EMD Millipore, Billerica, MA) and electroporated into E. cloni supreme cells (Lucigen, Middleton, WI). Libraries were expanded in culture and miniprepped (Qiagen, Valencia, CA) to roughly 100 ng/μl aliquots. 1 g of library was drop dialyzed and electroporated into C321.ΔA.Nendint for subsequent FACS experiments. Colony counts on appropriate antibiotic containing plates within one doubling time after transformation revealed library sizes of roughly 1×10⁶ for AARS libraries in Ecloni hosts and 1×10⁷ in C321.ΔA.Nendint hosts.

### Flow Cytometry and Cell Sorting

AARS libraries were subject to three rounds of fluorescence activated sorting in a Beckman Coulter MoFlo Astrios. Prior to each round, the usual NSAA incorporation assay procedure was followed such that cells would express GFP reporter proportional to the activity of the AARS library member. One notable deviation from that procedure was the use of a higher and variable inoculum volume to screen the full library at each stage. Cells displaying the top 0.5% of fluorescence activation (50 k cells) were collected after Round 1, expanded overnight, and used to inoculate experimental cultures for the next round. Because the next round was a negative screening round, the desired NSAA was not added into culture medium. The rest of the NSAA incorporation assay procedure was followed in order to eliminate cells that remained fluorescence due to promiscuous AARS activity on standard amino acids. In the second sort, cells displaying the lowest 10% of visible fluorescence (500 k cells) were collected. Cells passing the second round were expanded overnight and used to inoculate the third and final round of sorting. The experimental cultures for the third round were treated as the first round and were sorted for the upper 0.05% of fluorescence activation (1 k cells). The final cells collected were expanded overnight and plated for sequencing and downstream testing. Libraries were frozen at each stage before and after sorting. FlowJo X software was used to analyze the flow cytometry data. Constructs of interest were grown overnight, miniprepped, and transformed into C321.ΔA.Ubiq-UAG-sfGFP for further analysis in plate reader assays.

### EXAMPLE VI

### Embodiments

The present disclosure provides a method of making a target polypeptide in a cell, wherein the target polypeptide includes a non-standard amino acid (NSAA) substitution at an amino acid target location, including genetically modifying the cell to express the target polypeptide including a non-standard amino acid substitution at an amino acid target location using an engineered amino-acyl tRNA synthetase and transfer RNA pair corresponding to the non-standard amino acid, and wherein the cell expresses the target polypeptide including a standard amino acid or undesired NSAA at the amino acid target location when the engineered amino-acyl tRNA synthetase and transfer RNA pair non-selectively adds the standard amino acid or undesired NSAA at the amino acid target location, wherein a removable protecting group is attached to the target polypeptide adjacent to the amino acid target location, such that when the removable protecting group is removed, an N-end amino acid is exposed at the amino acid target location. According to one aspect, the removable protecting group is a cleavable protecting group that is orthogonal within the cell. According to one aspect, the removable protecting group is an enzyme cleavable protecting group. According to one aspect, the removable protecting group is a protein that is cleavable by a corresponding enzyme. According to one aspect, the removable protecting group is ubiquitin that is cleavable by Ubp1. According to one aspect, the cell is genetically modified to include a foreign nucleic acid sequence encoding the target polypeptide including a non-standard amino acid substitution at an amino acid target location and a removable protecting group attached to the target polypeptide adjacent to the amino acid target location. According to one aspect, a detectable moiety is attached to the C-end of the target polypeptide. According to one aspect, a detectable moiety is attached to the C-end of the target polypeptide, wherein the detectable moiety is a fluorescent moiety. According to one aspect, a detectable moiety is attached to the C-end of the target polypeptide, wherein the detectable moiety is a reporter protein. According to one aspect, the cell is genetically modified to include a foreign nucleic acid sequence encoding the target polypeptide including a non-standard amino acid substitution at an amino acid target location, a removable protecting group attached to the target polypeptide adjacent to the amino acid target location and a detectable moiety attached to the C-end of the target polypeptide, wherein the nonstandard amino acid is encoded by a corresponding nonsense or sense codon. According to one aspect, the cell is genetically modified to include a foreign nucleic acid sequence encoding an amino-acyl tRNA synthetase and a transfer RNA corresponding to the nonstandard amino acid and wherein the nonstandard amino acid is provided to the cell and the cell expresses the synthetase and the transfer RNA to include the nonstandard amino acid at the amino acid target location. According to one aspect, the cell is genetically modified to include a foreign nucleic acid sequence encoding an enzyme for cleaving the removable protecting group under influence of a constitutive or an inducible promoter. According to one aspect, the cell includes an adapter protein that coordinates with a protease for degrading the target polypeptide when the N-end amino acid is a standard amino acid or an undesired NSAA. According to one aspect, the cell includes an adapter protein that coordinates with a protease for degrading the target polypeptide when the N-end amino acid is a standard amino acid or an undesired NSAA, wherein the adapter protein is under influence of a constitutive or an inducible promoter. According to one aspect, the method further includes the cell expressing an enzyme that cleaves the removable protecting group to generate an N-end amino acid. According to one aspect, the method further includes the cell expressing an enzyme that cleaves the removable protecting group to generate an N-end amino acid, and the cell expressing a protease wherein the protease degrades the target polypeptide when the N-end amino acid is a standard amino acid or an undesired NSAA. According to one aspect, the method further includes the cell expressing an enzyme that cleaves the removable protecting group to generate an N-end amino acid, and the cell expressing a protease wherein the protease degrades the target polypeptide when the N-end amino acid is a standard amino acid or an undesired NSAA to thereby enrich the target polypeptide including a non-standard amino acid substitution within the cell. According to one aspect, the method further includes the cell expressing an enzyme that cleaves the removable protecting group to generate an N-end amino acid, and the cell expressing an adapter protein that coordinates with a protease, wherein the protease degrades the target polypeptide when the N-end amino acid is a standard amino acid or an undesired NSAA. According to one aspect, the method further includes the cell expressing an enzyme that cleaves the removable protecting group to generate an N-end amino acid, and the cell expressing an adapter protein that coordinates with a protease, wherein the protease degrades the target polypeptide when the N-end amino acid is a standard amino acid or an undesired NSAA, and wherein the adapter protein is under influence of an inducible promoter. According to one aspect, the method further includes the cell expressing an enzyme that cleaves the removable protecting group to generate an N-end amino acid, and the cell expressing a ClpS-ClpAP protease system wherein the ClpS-ClpAP protease system degrades the target polypeptide when the N-end amino acid is a standard amino acid or an undesired NSAA to thereby enrich the target polypeptide including a desired non-standard amino acid substitution within the cell. According to one aspect, the method further includes the cell expressing an enzyme that cleaves the removable protecting group to generate an N-end amino acid, and the cell expressing a ClpS-ClpAP protease system wherein the ClpS-ClpAP protease system degrades the target polypeptide when the N-end amino acid is a standard amino acid or an undesired NSAA to thereby enrich the target polypeptide including a desired non-standard amino acid substitution within the cell, and wherein the ClpS protein is a natural homolog or a ClpS_V65I mutant. According to one aspect, the method further includes a detectable moiety is attached to the C-end of the target polypeptide and further including the cell expressing an enzyme that cleaves the removable protecting group to generate an N-end amino acid, and the cell expressing an adapter protein for a protease, wherein the protease degrades the target polypeptide when the N-end amino acid is a standard amino acid or an undesired NSAA to thereby enrich the target polypeptide including a desired non-standard amino acid substitution within the cell, and detecting the detectable moiety as a measure of the amount of the target polypeptide including a non-standard amino acid substitution within the cell. According to one aspect, the cell is a prokaryotic cell or a eukaryotic cell. According to one aspect, the cell is a microorganism such as a bacterium. According to one aspect, the cell is E. coli. According to one aspect, the cell is a genetically modified E. coli.

The disclosure provides a method of designing an amino acyl tRNA synthetase variant for preferential selection of a desired non-standard amino acid against its standard amino acid counterpart or undesired NSAAs for incorporation into a protein in a cell including genetically modifying the cell to express the target polypeptide including a non-standard amino acid substitution at an amino acid target location using an engineered amino-acyl tRNA synthetase and transfer RNA pair corresponding to the non-standard amino acid or undesired NSAA, and wherein the cell expresses the target polypeptide including a standard amino acid or undesired NSAA at the amino acid target location when the engineered amino-acyl tRNA synthetase and transfer RNA pair non-selectively adds the standard amino acid or undesired NSAA at the amino acid target location, wherein a removable protecting group is attached to the target polypeptide adjacent to the amino acid target location, such that when the removable protecting group is removed, an N-end amino acid is exposed at the amino acid target location, and wherein a detectable moiety is attached to the C-end of the target polypeptide, wherein the cell is genetically modified to include a foreign nucleic acid sequence encoding an amino-acyl tRNA synthetase and a transfer RNA corresponding to the nonstandard amino acid and wherein the nonstandard amino acid is provided to the cell and the cell expresses the synthetase and the transfer RNA to include the nonstandard amino acid at the amino acid target location, the cell expressing an enzyme that cleaves the removable protecting group to generate an N-end amino acid, and the cell expressing an adapter protein for a protease, wherein the protease degrades the target polypeptide when the N-end amino acid is a standard amino acid or undesired NSAA to thereby enrich the target polypeptide including a desired non-standard amino acid substitution within the cell, detecting the detectable moiety as a measure of the amount of the target polypeptide including a non-standard amino acid substitution within the cell, and repeatedly testing a modified synthetase in the genetically modified cell for improved production of the target polypeptide including a non-standard amino acid substitution.

The disclosure provides an engineered cell including a foreign nucleic acid sequence encoding a target polypeptide including a non-standard amino acid substitution at an amino acid target location and a removable protecting group attached to the target polypeptide adjacent to the amino acid target location.

The disclosure provides an engineered cell including a foreign nucleic acid sequence encoding a target polypeptide including a non-standard amino acid substitution at an amino acid target location, a removable protecting group attached to the target polypeptide adjacent to the amino acid target location and a detectable moiety attached to the C-end of the target polypeptide.

The disclosure provides an engineered cell including (a) a foreign nucleic acid sequence encoding a target polypeptide including a non-standard amino acid substitution at an amino acid target location, a removable protecting group attached to the target polypeptide adjacent to the amino acid target location and a detectable moiety attached to the C-end of the target polypeptide; (b) a foreign nucleic acid sequence encoding an amino-acyl tRNA synthetase and a transfer RNA corresponding to the nonstandard amino acid; (c) an adapter protein for a protease for degrading the target polypeptide having a standard amino acid or undesired NSAA as the N-end amino acid, wherein the adapter protein is under influence of a constitutive promoter or an inducible promoter.

The disclosure provides a nucleic acid construct encoding a target polypeptide including a non-standard amino acid substitution at an amino acid target location and a removable protecting group attached to the target polypeptide adjacent to the amino acid target location.

The disclosure provides a nucleic acid construct encoding a target polypeptide including a non-standard amino acid substitution at an amino acid target location, a removable protecting group attached to the target polypeptide adjacent to the amino acid target location and a detectable moiety attached to the C-end of the target polypeptide.

The disclosure provides a nucleic acid construct encoding adapter protein Clps or mutants or variants thereof.

The disclosure provides a nucleic acid construct encoding ClpS_V65I mutant.

The above disclosure generally describes the present invention. All references disclosed herein are expressly incorporated by reference. A more complete understanding can be obtained by reference to the following specific examples which are provided herein for purposes of illustration only, and are not intended to limit the scope of the invention.

1. A method of making a target polypeptide in a cell, wherein the target polypeptide includes a non-standard amino acid (NSAA) substitution at an amino acid target location, comprising
genetically modifying the cell to express the target polypeptide including a non-standard amino acid substitution at an amino acid target location using an engineered amino-acyl tRNA synthetase and transfer RNA pair corresponding to the non-standard amino acid, and wherein the cell expresses the target polypeptide including a standard amino acid or undesired NSAA at the amino acid target location when the engineered amino-acyl tRNA synthetase and transfer RNA pair non-selectively adds the standard amino acid or undesired NSAA at the amino acid target location,
wherein a removable protecting group is attached to the target polypeptide adjacent to the amino acid target location, such that when the removable protecting group is removed, an N-end amino acid is exposed at the amino acid target location.

2. The method of claim 1 wherein the removable protecting group is a cleavable protecting group that is orthogonal within the cell.

3. The method of claim 1 wherein the removable protecting group is an enzyme cleavable protecting group.

4. The method of claim 1 wherein the removable protecting group is a protein that is cleavable by a corresponding enzyme.

5. The method of claim 1 wherein the removable protecting group is ubiquitin that is cleavable by Ubp1.

6. The method of claim 1 wherein the cell is genetically modified to include a foreign nucleic acid sequence encoding the target polypeptide including a non-standard amino acid substitution at an amino acid target location and a removable protecting group attached to the target polypeptide adjacent to the amino acid target location.

7. The method of claim 1 wherein a detectable moiety is attached to the C-end of the target polypeptide.

8. The method of claim 1 wherein a detectable moiety is attached to the C-end of the target polypeptide, wherein the detectable moiety is a fluorescent moiety or a reporter protein.

9. (canceled)

10. The method of claim 1 wherein the cell is genetically modified to include a foreign nucleic acid sequence encoding the target polypeptide including a non-standard amino acid substitution at an amino acid target location, a removable protecting group attached to the target polypeptide adjacent to the amino acid target location and a detectable moiety attached to the C-end of the target polypeptide, wherein the nonstandard amino acid is encoded by a corresponding nonsense or sense codon.

11. The method of claim 1 wherein the cell is genetically modified to include a foreign nucleic acid sequence encoding an amino-acyl tRNA synthetase and a transfer RNA corresponding to the nonstandard amino acid and wherein the nonstandard amino acid is provided to the cell and the cell expresses the synthetase and the transfer RNA to include the nonstandard amino acid at the amino acid target location.

12. The method of claim 1 wherein the cell is genetically modified to include a foreign nucleic acid sequence encoding an enzyme for cleaving the removable protecting group under influence of a constitutive or an inducible promoter.

13. The method of claim 1 wherein the cell includes an adapter protein that coordinates with a protease for degrading the target polypeptide when the N-end amino acid is a standard amino acid or an undesired NSAA.

14. The method of claim 1 wherein the cell includes an adapter protein that coordinates with a protease for degrading the target polypeptide when the N-end amino acid is a standard amino acid or an undesired NSAA, wherein the adapter protein is under influence of a constitutive or an inducible promoter.

15. (canceled)

16. (canceled)

17. The method of claim 1 further comprising
the cell expressing an enzyme that cleaves the removable protecting group to generate an N-end amino acid, and
the cell expressing a protease wherein the protease degrades the target polypeptide when the N-end amino acid is a standard amino acid or an undesired NSAA to thereby enrich the target polypeptide including a non-standard amino acid substitution within the cell.

18. (canceled)

19. The method of claim 1 further comprising
the cell expressing an enzyme that cleaves the removable protecting group to generate an N-end amino acid, and
the cell expressing an adapter protein that coordinates with a protease, wherein the protease degrades the target polypeptide when the N-end amino acid is a standard amino acid or an undesired NSAA, and wherein the adapter protein is under influence of an inducible promoter.

20. (canceled)

21. The method of claim 1 further comprising
the cell expressing an enzyme that cleaves the removable protecting group to generate an N-end amino acid, and
the cell expressing a ClpS-ClpAP protease system wherein the ClpS-ClpAP protease system degrades the target polypeptide when the N-end amino acid is a standard amino acid or an undesired NSAA to thereby enrich the target polypeptide including a desired non-standard amino acid substitution within the cell, and wherein the ClpS protein is a natural homolog or a ClpS_V65I mutant.

22. The method of claim 1 wherein a detectable moiety is attached to the C-end of the target polypeptide and further comprising
the cell expressing an enzyme that cleaves the removable protecting group to generate an N-end amino acid, and
the cell expressing an adapter protein for a protease, wherein the protease degrades the target polypeptide when the N-end amino acid is a standard amino acid or an undesired NSAA to thereby enrich the target polypeptide including a desired non-standard amino acid substitution within the cell, and
detecting the detectable moiety as a measure of the amount of the target polypeptide including a non-standard amino acid substitution within the cell.

23.-25. (canceled)

26. A method of designing an amino acyl tRNA synthetase variant for preferential selection of a desired non-standard amino acid against its standard amino acid counterpart or undesired NSAAs for incorporation into a protein in a cell comprising
genetically modifying the cell to express the target polypeptide including a non-standard amino acid substitution at an amino acid target location using an engineered amino-acyl tRNA synthetase and transfer RNA pair corresponding to the non-standard amino acid or undesired NSAA, and wherein the cell expresses the target polypeptide including a standard amino acid or undesired NSAA at the amino acid target location when the engineered amino-acyl tRNA synthetase and transfer RNA pair non-selectively adds the standard amino acid or undesired NSAA at the amino acid target location,
wherein a removable protecting group is attached to the target polypeptide adjacent to the amino acid target location, such that when the removable protecting group is removed, an N-end amino acid is exposed at the amino acid target location, and wherein a detectable moiety is attached to the C-end of the target polypeptide,
wherein the cell is genetically modified to include a foreign nucleic acid sequence encoding an amino-acyl tRNA synthetase and a transfer RNA corresponding to the nonstandard amino acid and wherein the nonstandard amino acid is provided to the cell and the cell expresses the synthetase and the transfer RNA to include the nonstandard amino acid at the amino acid target location,
the cell expressing an enzyme that cleaves the removable protecting group to generate an N-end amino acid, and
the cell expressing an adapter protein for a protease, wherein the protease degrades the target polypeptide when the N-end amino acid is a standard amino acid or undesired NSAA to thereby enrich the target polypeptide including a desired non-standard amino acid substitution within the cell,
detecting the detectable moiety as a measure of the amount of the target polypeptide including a non-standard amino acid substitution within the cell, and
repeatedly testing a modified synthetase in the genetically modified cell for improved production of the target polypeptide including a non-standard amino acid substitution.

27.-28. (canceled)

29. An engineered cell including
(a) a foreign nucleic acid sequence encoding a target polypeptide including a non-standard amino acid substitution at an amino acid target location, a removable protecting group attached to the target polypeptide adjacent to the amino acid target location and a detectable moiety attached to the C-end of the target polypeptide;
(b) a foreign nucleic acid sequence encoding an amino-acyl tRNA synthetase and a transfer RNA corresponding to the nonstandard amino acid;
(c) an adapter protein for a protease for degrading the target polypeptide having a standard amino acid or undesired NSAA as the N-end amino acid, wherein the adapter protein is under influence of a constitutive promoter or an inducible promoter.

30.-32. (canceled)

33. A nucleic acid construct encoding ClpS_V65I mutant.
